(19) United States
(12) Patent Application Publication
(10) Pub. No.: US 2004/0214176 A1
(43) Pub. Date: Oct. 28, 2004

(54) Multiplexed DNA assays using structure-specific endonucleases

(76) Inventors: James C. Osborne, Orange, CA (US); John P. Wareham, Orange, CA (US); Stephen L. Pentoney JR., Chino Hills, CA (US); Martin F. Siaw, Irvine, CA (US)

Correspondence Address: SHELDON & MAK, INC, 225 SOUTH LAKE AVENUE, 9TH FLOOR, PASADENA, CA, 91101, US

(21) Appl. No.: 10/420,322

(22) Filed: Apr. 22, 2003

(51) Int. Cl. C12Q 1/68; C12P 19/34

(52) U.S. Cl. 435/6; 435/91.2

(57) ABSTRACT

Oligonucleotide probes, kits, and methods useful for detecting more than one target nucleic acid at a time are provided. The invention provides oligonucleotides that interact with target nucleic acids to form first stage cleavage structures that are cleaved by a structure-specific nuclease. In an embodiment, the cleavage products of the first stage cleavage reactions interact with secondary cleavage oligonucleotides that are specific for each target nucleic acid and that are coupled to a solid support. The cleavage of the specific secondary cleavage oligonucleotides leads to detectible changes in fluorescence and is used to detect multiple target nucleic acids in one assay. In one embodiment, the solid support is a coded bead.

### BACKGROUND

Numerous assay methods have been developed for the detection and characterization of specific target nucleic acids and sequence variations within nucleic acids of interest. Demand has increased for nucleic acid assays due to the availability of nucleic acid sequence data from the human genome, as well as sequence data from a variety of organisms contributing to human diseases. It is important that these assays be sensitive enough to detect target nucleic acids from samples containing limited copies of the target nucleic acid of interest. Another aspect that has become important, but which has yet to be effectively implemented, is to be able to analyze numerous target nucleic acids at the same time and in the same reaction.

In one assay used to detect the presence of specific nucleic acids, a pair of oligonucleotides interact with a target nucleic acid to form a cleavage structure that is cleaved by a structure-specific nuclease (see Hall et al. U.S. Pat. No. 5,994,069). The resulting cleaved and/or uncleaved oligonucleotides are then analyzed and resolved. A problem with existing implementations of this type of assay is that they are limited in multiplexing capacity. Although it is possible to increase the multiplexing capacity of this assay by designing unique fluorescently labeled probes for each assay, a unique fluorescent label for each assay would be required in order to unambiguously assign a fluorescent signal to the specific assay that generated the release of the labeled product. Practical limitations in the number of commercially available fluorescent labels that could be spectrally resolved simultaneously severely limit the multiplexing capacity of the assay. In addition, the use of a multitude of fluorescent probes would greatly increase the cost and complexity of using this type of detection assay.

A need, therefore, exists for an analytical detection system that is able to detect multiple target nucleic acids in one reaction in an assay format that is also sensitive, fast, reliable, and cost-effective.

### SUMMARY

The present invention satisfies this need. The present invention provides a fast, reliable, and cost-effective system and method for detecting multiple target nucleic acids in one reaction or assay.

In one aspect, the invention encompasses a method of detecting the presence of more than one target nucleic acid molecule. A sample having at least one target nucleic acid is provided, each target nucleic acid has a first region, a second region and a third region. The first region is contiguous to and downstream from the second region and the second region is contiguous to and downstream from the third region.

A primary cleavage oligonucleotide corresponding to each target nucleic acid is also provided. Each primary cleavage oligonucleotide has a 3′ portion and a 5′-cleavage portion. The 3′ portion of each primary cleavage oligonucleotide comprises a sequence complementary to the third region of each corresponding target nucleic acid. The cleavage portion of each primary cleavage oligonucleotide comprises a sequence complementary to the second region of each corresponding target nucleic acid.

An invading oligonucleotide corresponding to each target nucleic acid is also provided. Each invading oligonucleotide has a 3′ portion and a 5′ portion. The 3′ portion of each invading oligonucleotide has a sequence complementary to the second region of each corresponding target nucleic acid. The 5′ portion of each invading oligonucleotide has a sequence complementary to the first region of each corresponding target nucleic acid. A nucleic acid cleavage means is also provided.

A primary cleavage structure is generated for each target nucleic acid, wherein at least the 3′ portion of the primary cleavage oligonucleotide is annealed to a corresponding target nucleic acid and at least the 5′ portion of the invading oligonucleotide is annealed to a corresponding target nucleic acid. Cleavage of the primary cleavage structure is performed by the nucleic acid cleavage means to cleave each primary cleavage oligonucleotide to generate a cleaved assay specific probe corresponding to each target nucleic acid. Each cleaved assay specific probe has a 5′ region and a 3′ region.

A secondary cleavage oligonucleotide is provided for each target nucleic acid. Each secondary cleavage oligonucleotide has contiguous first and second regions. The first region has a 3′ end of the secondary cleavage oligonucleotide. The 3′ end of the secondary cleavage oligonucleotide is coupled to a solid support. The second region comprises a 5′ end of the secondary cleavage oligonucleotide and a region of self-complementarity. The second region is coupled to a detectible label in a first state. The first region of each secondary cleavage oligonucleotide is complementary to a cleaved assay specific probe for a corresponding target nucleic acid. A portion of the second region of each secondary cleavage oligonucleotide is complementary to at least a portion of the 3′ region of the corresponding cleaved assay specific probe.

A secondary cleavage structure is generated, wherein the 5′ end of each secondary cleavage oligonucleotide is cleaved from the remainder of the second region of each secondary cleavage oligonucleotide changing the detectible label to a second state. Cleavage of secondary cleavage oligonucleotides is detected by a change in fluorescence, thereby detecting the target nucleic acid corresponding to the secondary cleavage oligonucleotide.

In a first additional embodiment, the secondary cleavage oligonucleotide has contiguous first and second regions. The first region has the 3′ end of the secondary cleavage oligonucleotide and is complementary to a cleaved assay specific probe for a corresponding target nucleic acid. The second region has a region of self-complementarity and a cleavage portion. The cleavage portion is coupled to a detectible label. A portion of the second region of each secondary cleavage oligonucleotide is complementary to at least a portion of the 3′ region of the corresponding cleaved assay specific probe.

A secondary cleavage structure is generated, wherein the cleavage portion of each secondary cleavage oligonucleotide is cleaved from the remainder of the second region of each secondary cleavage oligonucleotide. A capture oligonucleotide is provided for each secondary cleavage oligonucleotide comprising a sequence complementary to the cleavage portion of the secondary cleavage oligonucleotide, the capture oligonucleotide being coupled to a distinct dye coded bead. The cleavage products of secondary cleavage oligonucleotides are detected by detecting a change in fluorescence of the dye coded bead, thereby detecting the target nucleic acid corresponding to each capture oligonucleotide.

In a second additional embodiment, a template oligonucleotide is provided. The template oligonucleotide has a first region and a second region. The first region of each template oligonucleotide is complementary to at least a portion of the cleavage products of secondary cleavage oligonucleotides. The second region of each template oligonucleotide is complementary to a target-specific reporter oligonucleotide coupled to a distinct coded bead.

A ligation means is provided. A ligation complex is generated where the first region of each template oligonucleotide is annealed to the corresponding cleavage products of secondary cleavage oligonucleotides, and the second region of each template oligonucleotide is annealed to the corresponding target-specific reporter oligonucleotide. The cleavage products of secondary cleavage oligonucleotides are then ligated to the corresponding target-specific reporter oligonucleotide to form target-specific detection complexes. The target-specific detection complexes are detected by a change in the fluorescence of the distinct coded bead, thereby detecting each corresponding target nucleic acid.

In a third additional embodiment, a 3′ end of the primary cleavage oligonucleotide is coupled to a solid support and to a detectible label in a first state (i.e., quenched state). Cleavage of the primary cleavage structure by the nucleic acid cleavage means changes the detectible label to a second state (i.e., unquenched state). Cleavage of the primary cleavage oligonucleotide is detected by a change in fluorescence, thereby detecting the target nucleic acid corresponding to the primary cleavage oligonucleotide.

In a fourth additional embodiment, a 5′ end of the primary cleavage oligonucleotide is coupled to a solid support and to a detectible label in a first state. Cleavage of the primary cleavage structure by the nucleic acid cleavage means changes the detectible label to a second state. Cleavage of the primary cleavage oligonucleotide is detected by a change in fluorescence, thereby detecting the target nucleic acid corresponding to the primary cleavage oligonucleotide.

In a fifth additional embodiment, a primary cleavage oligonucleotide has a cleavage portion coupled to a detectible label. The cleavage portion and the detectible label are cleaved by the nucleic acid cleavage means. A capture oligonucleotide is provided for each primary cleavage oligonucleotide comprising a sequence complementary to the cleavage portion of the primary cleavage oligonucleotide. The capture oligonucleotide is coupled to a distinct dye coded bead. Cleavage of primary cleavage oligonucleotides is detected by a change in the fluorescence of the dye coded beads, thereby detecting the target nucleic acid corresponding to each capture oligonucleotide.

The present invention is also directed to a kit for detecting the presence of a target nucleic acid. The kit includes primary cleavage oligonucleotides corresponding to the wild type and mutant alleles of the target nucleic acid, an invading oligonucleotide corresponding to the target nucleic acid, a nucleic acid cleavage means for cleaving the primary cleavage structure, formed by the primary cleavage oligonucleotide, the invading oligonucleotide, and the appropriate target nucleic acid, to form a cleaved assay specific probe. The kit also includes secondary cleavage oligonucleotides. The secondary cleavage oligonucleotides are coupled to a solid support and to a detectible label in a first state. The secondary cleavage oligonucleotides have a portion complementary to at least a portion of the cleaved assay specific probe. The presence of the cleaved assay specific probe causes cleavage of a portion of the secondary cleavage oligonucleotide, thereby changing the detectible label to a second state. The kit contains a detector for detecting cleavage of the secondary cleavage oligonucleotide by a change in fluorescence, thereby detecting the target nucleic acid corresponding to the secondary cleavage oligonucleotide.

### THE DRAWINGS

These features, aspects and advantages of the present invention will become better understood with regard to the following description, appended claims and accompanying figures where:

FIG. 1 is a schematic illustration of a preferred embodiment of the system and method for detecting a target nucleic acid molecule of the present invention;

FIG. 2 is a schematic illustration of a flow cytometry system that can be used in the present invention;

FIG. 3 is a flow chart illustrating a method for analyzing fluorescence changes in beads according to an embodiment of the present invention;

FIG. 4 is an illustration of a primary cleavage structure according to an exemplary embodiment of the present invention;

FIG. 5 is an illustration of a cleaved assay specific probe hybridizing to a secondary cleavage oligonucleotide;

FIG. 6 is a schematic illustration of a first additional embodiment of the present invention where a secondary assay specific probe is cleaved from a secondary cleavage oligonucleotide and then hybridized to a capture oligonucleotide coupled to a dye encoded bead;

FIG. 7 is a schematic illustration of a first additional embodiment of the present invention where a secondary assay specific probe is cleaved from a secondary cleavage oligonucleotide and then hybridized along with a capture oligonucleotide to a hyrbridization template oligonucletoide;

FIG. 8 is a schematic illustration of a second additional embodiment of the present invention where a secondary assay specific probe is cleaved from a secondary cleavage oligonucleotide and then ligated to a capture oligonucleotide coupled to a dye encoded bead;

FIG. 9 is a schematic illustration of a second additional embodiment of the present invention where the capture oligonucleotide has a hairpin loop;

FIG. 10 is a schematic illustration of a third additional embodiment of the present invention where a 3′ end of the primary cleavage oligonucleotide is coupled to a dye encoded bead;

FIG. 11 is an illustration of a primary cleavage oligonucleotide coupled at a 3′ end to a dye encoded bead according to the third additional embodiment of the present invention;

FIG. 12 is a schematic illustration of a cleavage portion of a primary cleavage oligonucleotide coupled to a dye encoded bead according to the fourth additional embodiment of the present invention; and

FIG. 13 is a schematic illustration of a fifth additional embodiment of the present invention where a cleaved primary assay specific probe is hybridized to a capture oligonucleotide coupled to a dye encoded bead.

### DESCRIPTION

The following discussion describes embodiments of the invention and several variations of these embodiments. This discussion should not be construed, however, as limiting the invention to those particular embodiments. Practitioners skilled in the art will recognize numerous other embodiments as well. In all of the embodiments described herein that are referred to as being preferred or particularly preferred, these embodiments are not essential even though they may be preferred.

Definitions

The terms “polynucleotide” or “nucleic acid” and their respective plurals are used essentially interchangeably herein and are intended to include naturally occurring or synthesized double stranded deoxyribonucleic acid (hereinafter “DNA”), single stranded DNA, or ribonucleic acid (hereinafter “RNA”).

The terms “complementary” or “complementarity” as used herein refer to polynucleotides that undergo Watson-Crick base pairing. Such base pairing also comprehends the pairing of “nucleoside analogs”, such as deoxyinosine, nucleosides with 2-aminopurine bases, and the like, that may be employed. As used herein, “nucleoside” includes the natural nucleosides, including 2′-deoxy and 2′-hydroxyl forms, e.g. as described in Komberg and Baker, DNA Replication, 2nd Ed. (Freeman, San Francisco, 1992). “Analogs” in reference to nucleosides includes synthetic nucleosides having modified base moieties and/or modified sugar moieties that are capable of specific hybridization, e.g. described by Scheit, Nucleotide Analogs (John Wiley, New York, 1980); Uhlman and Peyman, Chemical Reviews, 90:543-584 (1990), or the like. Such analogs include synthetic nucleosides designed to enhance binding properties, reduce degeneracy, increase specificity, and the like.

Complementarity need not be perfect; stable duplexes may contain mismatched base pairs or unmatched bases. Accordingly, two polynucleotides undergoing base pairing may be “partially complementary” or “totally or completely complementary”. “Totally complementary” as used herein means that the polynucleotide or oligonucleotide strands making up a duplex form a double stranded structure with one another such that every nucleotide in each strand undergoes Watson-Crick base pairing with a nucleotide in the other strand and that there are no mismatches. “Partially complementary” as used herein includes polynucleotide or oligonucleotide strands containing unmatched and non-hybridizing nucleotides, but in which the remaining “matched” nucleotides undergo Watson-Crick base pairing. Those skilled in the art can readily determine duplex stability empirically by considering a number of variables including, for example, the length of the oligonucleotide, base composition and sequence of the oligonucleotide, ionic strength and incidence of mismatched base pairs. The complement of a nucleic acid sequence as used herein refers to an polynucleotide which, when aligned with the nucleic acid sequence such that the 5′ end of one sequence is paired with the 3′ end of the other, is in “antiparallel association.”

The term “self-complementarity” when used in reference to a nucleic acid (e.g., an oligonucleotide) means that a nucleic acid can engage in intramolecular base-pairing where separate regions of the nucleic acid are capable of base-pairing with one another. Regions within primary cleavage oligonucleotides are considered to be self-complementarity when they may form a duplex of at least 3 contiguous base pairs with complete complementarity or when they may form a longer duplex that is partially complementary. Self-complementary as defined herein includes the ability of an oligonucleotide having a self-complementary region to successfully serve as both a target strand for a primary cleavage oligonucleotide, and as an upstream oligonucleotide that facilitates invasive cleavage of that primary cleavage oligonucleotide.

“Homology” refers to a degree of identity between two or more nucleic acid sequences. There may be partial homology or complete homology. A partially identical sequence is one that is less than 100% identical to another sequence.

“Hybridization” is used herein in reference to the pairing of complementary nucleic acids. Hybridization and the strength of hybridization is affected by variables such as the degree of complementary between the nucleic acids, stringency of the hybridization conditions involved, the Tₘ (melting temperature) of the formed hybrid, and the G:C ratio within the nucleic acids.

“Stringency” as used herein refers to the conditions of temperature, ionic strength, and the presence of other compounds, under which nucleic acid hybridizations are conducted. Under “high stringency” conditions, nucleic acid base pairing will only occur between nucleic acid fragments that have a high frequency of complementary base sequences. Conversely, nucleic acids which are not completely complementary to one another are typically able to hybridize and anneal together under conditions of “weak” or “low” stringency.

The term “oligonucleotide” as used herein is defined as a molecule comprised of two or more deoxyribonucleotides or ribonucleotides, preferably at least 5 nucleotides, more preferably at least about 10-15 nucleotides, and more preferably at least about 15 to 30 nucleotides. Such oligonucleotides may be generated by various methods, including chemical synthesis, DNA replication, reverse transcription, or a combination thereof.

A nucleic acid sequence, even if it is a sub-portion of a larger sequence, is understood to have 5′ and 3′ ends. This reflects the fact that mononucleotides are reacted to make oligonucleotides such that the 5′ phosphate of one mononucleotide pentose ring is attached to the 3′ oxygen of its neighbor in one direction via a phosphodiester linkage. A terminus or end of an oligonucleotide is referred to as the “5′end” if its 5′ phosphate is not linked to the 3′ oxygen of a mononucleotide pentose ring. A terminus of an oligonucleotide is referred to as the “3′ end” if its 3′ oxygen is not linked to a 5′ phosphate of a subsequent mononucleotide pentose ring.

As referred to herein, a first region along a nucleic acid strand is said to be “upstream” of another region if the 3′ end of the first region is before the 5′ end of the second “downstream” region when moving along a strand of nucleic acid in a 5′ to 3′ direction.

“Target nucleic acid” as used herein may comprise single or double-stranded DNA or RNA. Target nucleic acids contain a sequence that has at least partial complementarity with at least a primary cleavage oligonucleotide, and may also have at least partial complementarity with an invading oligonucleotide.

“Invading oligonucleotide” refers to an oligonucleotide which contains sequences at its 3′ end which are substantially the same as sequences located at the 5′ end of the non-flap portion of a primary cleavage oligonucleotide; these regions will compete for hybridization to the same segment along a complementary target nucleic acid.

A “capture oligonucleotide” as used herein refers to an oligonucleotide that is complementary and capable of hybridizing to at least a portion of another oligonucleotide or nucleic acid. Capture oligonucleotides can be immobilized to a solid support such that an oligonucleotide or nucleic acid that binds to the capture oligonucleotide becomes bound to the solid support.

The terms “ligating” or “ligation” refers to the formation of a phosphodiester bond between a 3′-OH and a 5′-P located at the termini of two strands of nucleic acid. The term “ligation means” refers to any agent capable of facilitating a ligation between two nucleic acids, including for example, DNA ligases and RNA ligases.

A “linkage oligonucleotide” as used herein refers to an oligonucleotide or larger nucleic acid that is complementary and capable of annealing with two oligonucleotides that eventually become ligated together. Upon hybridization, a “ligation complex” is formed between the linkage oligonucleotide and two other oligonucleotides that places the 5′ end of one oligonucleotide in proximity with the 3′ end of the other oligonucleotide and facilitates the formation of a phosphodiester bond by the ligation means.

An oligonucleotide is said to be present in “excess” relative to another nucleic acid molecule if that oligonucleotide is present at a higher molar concentration than the other nucleic acid molecule. As an example, when a primary cleavage oligonucleotide is present in excess of a target nucleic acid it will be present in at least a 10 to 100-fold molar excess; typically at least 1 pmole of each primary cleavage oligonucleotide would be used when the target nucleic acid sequence is present at about 10 fmoles or less.

The term “nucleic acid sequence” as used herein refers to an oligonucleotide, nucleotide or polynucleotide, and fragments or portions thereof, and to DNA or RNA of genomic or synthetic origin which may be single or double-stranded, and represent the sense or antisense strand.

Overview

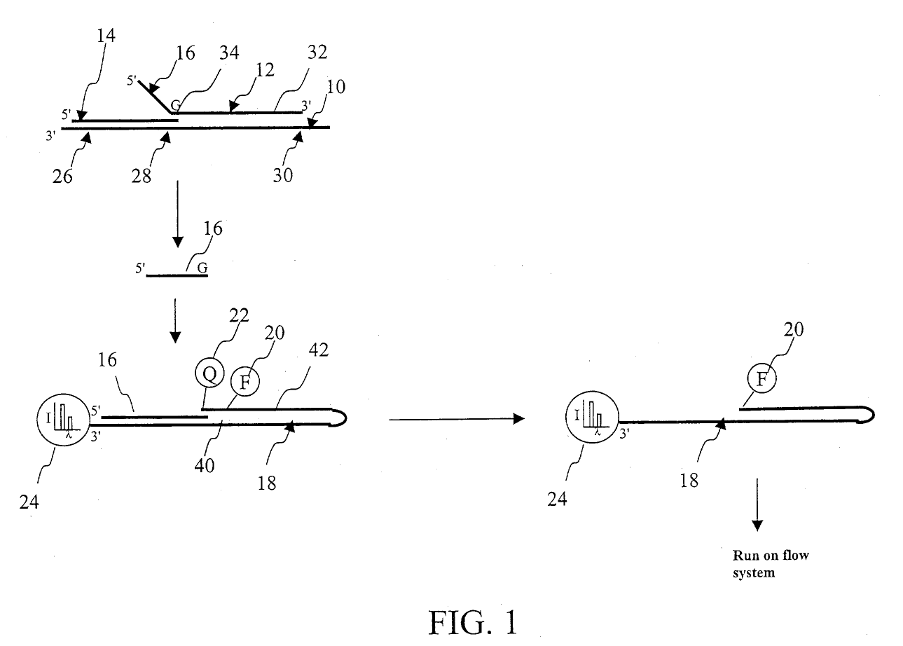

An overview of a system and method according to a preferred embodiment is shown in FIG. 1, and will now be described to illustrate the various components of the system. A target nucleic acid 10, is hybridized to a primary cleavage oligonucleotide 12. An invading oligonucleotide 14 also hybridizes to the target nucleic acid 10 to form a primary cleavage structure. A structure-specific nuclease cleaves the primary cleavage oligonucleotide 12, releasing a cleaved assay specific probe 16. The cleaved assay specific probe 16 hybridizes to a secondary cleavage oligonucleotide 18 having a fluor 20 and a quencher 22. The secondary cleavage oligonucleotide is coupled to a dye-encoded bead 24. Upon hybridization of the cleaved assay specific probe 16 to the secondary cleavage oligonucleotide 18, the quencher 20 is released allowing the fluor to be capable of fluorescent resonance energy transfer.

Target Nucleic Acids

The invention provides means for forming nucleic acid cleavage structures that are dependent upon the presence of a target nucleic acid. The nucleic acid cleavage structures are cleaved by a nucleic acid cleavage means to release distinct cleaved assay specific primary cleavage oligonucleotides corresponding to each target nucleic acid.

In a typical embodiment, one or more target nucleic acids are derived from one or more samples. Each sample is known to contain, or is suspected of containing, one or more target nucleic acids. Target nucleic acids include various types of both RNA and DNA (including single stranded DNA and double stranded DNA) that are obtained according to standard techniques known in the art. For example, samples containing nucleic acids may be obtained from a tissue sample, tissue culture cells, samples containing bacteria and/or viruses, etc. Also, the target nucleic acid may also be transcribed in vitro from a DNA template or may be synthetic, chemically synthesized, or generated in a PCR. Further, nucleic acids may be isolated from organisms in the form of genomic material, plasmids, or similar extrachromosomal DNA, or the nucleic acid may be a fragment of such material generated by treatment with a restriction endonuclease or other cleavage agent.

The binding of invading oligonucleotides 14 and primary cleavage oligonucleotides 12, described in more detail below, divides the target nucleic acid 10 into three distinct regions, as shown in FIG. 1. A first region 26, has complementarity to only the invading oligonucleotide 14; a second region 28, has complementarity to both the invading oligonucleotide 14 and the primary cleavage oligonucleotide 12; and a third region 30, has complementarity only to a portion of the corresponding primary cleavage oligonucleotide 12. The first region of each target nucleic acid 10 is contiguous to and downstream from the second region, and the second region of each target nucleic acid is contiguous to and downstream from the third region.

A target nucleic acid may be either single-stranded or double-stranded. Double-stranded target nucleic acids can be rendered single stranded, for example by heating. In the methods described herein, the target nucleic acid can be reused or recycled during multiple rounds of hybridization with oligonucleotides and cleavage means.

In a preferred embodiment, the methods of the invention are used to detect single nucleotide polymorphisms (SNP's). Exemplary targets include, but are not limited to, Apolipoprotein E (ApoE)(C112R); Apolipoprotein E (ApoE)(R158C); Factor II (G320210A); Factor V (Leiden)(G1691A); Glycoprotein Ia (Gpla)(C807T); Glycoprotein IIIa (PL A1/A2)(T565C); Mehtylenetetrahydrofolate Reductase (MTHFR) (A11298C); Methlenetetrahydrofolate Reductase (MTHFR) (C677T); Plasminogen Activator Inhibitor-I (PAL-1); ATP-Binding Cassette, Subfamily B, Member 1; Cytochrome P450, Subfamily 1A, Polypeptide 1; Cytochrome P450, Subfamily 1A, Polypeptide 2; Cytochrome P450, Subfamily 11B, Polypeptide 6; Cytochrome P450, Subfamily 11C, Polypeptide 9, Glucuronidase, Beta; Inrterferon, Gamma; Interleukin 1, Beta; Interleukin 2; Interleukin 4; Interleukin 6; Interleukin 8; Interleukin 10; Involucrin; Matrix Metalloproteinase 3; Nuclear Factor of Kappa Light Polypeptide Gene Enhancer in B-cells Inhibitor, Alpha; Nuclear Receptor Subfamily 3, Group C, Member 1; Oncostatin M; Prostaglandin-Endoperoxide Synthase 2; Small Inducible Cytokine A2; Solute Carrier Family 21(organic anion transporter), Member 9 (hSLC21A9); Sulfotransferase Family, Cytosolic, 1A, Phenol-Preferrinig, Members 1-3, Pan Detection; Transforming Growth Factor, Beta 1; Tumor Necrosis Factor (TNF Superfamily, Member 2); v-fos FBJ murine osteosarcoma viral oncogene homolog; and v-myc myelocytomatosis viral oncogene homolog (avian).

Primary Cleavage Structure

The methods described herein typically utilize at least a pair of oligonucleotides that interact with each target nucleic acid to form a primary cleavage structure that is a substrate for a structure-specific nuclease.

For each target nucleic acid 10, there is a corresponding primary cleavage oligonucleotide 12. As shown in FIG. 1, each primary cleavage oligonucleotide comprises a 3′ portion 32 and a cleavage portion 34. The 3′ portion 32 of each primary cleavage oligonucleotide defines the third region 30 of the target nucleic acid sequence by being the complement of that region for a corresponding target nucleic acid. The cleavage portion 34 of each primary cleavage oligonucleotide defines the second region 28 of the target nucleic acid sequence by being the complement of that region for a corresponding target nucleic acid. The target nucleic acid strand that anneals with the primary cleavage oligonucleotide does so in a typical anti-parallel orientation.

For each target nucleic acid, there is a corresponding invading oligonucleotide 14. Each invading oligonucleotide comprises 3′ and 5′ portions. The 3′ portion of an invading oligonucleotide comprises a sequence complementary to the second region 28 of the corresponding target nucleic acid 10. Thus, for each particular target nucleic acid 10, the 3′ portion of an invading oligonucleotide 14 overlaps with the cleavage portion 34 of the primary cleavage oligonucleotide. The invading oligonucleotide 14 is located upstream of the primary cleavage oligonucleotide 12 with respect to the target nucleic acid strand 10. Both the invading oligonucleotide 14 and the primary cleavage oligonucleotide 12 anneal to the corresponding target nucleic acid 10 in an anti-parallel orientation to the target nucleic acid strand. The 5′ portion of the invading oligonucleotide 14 comprises a sequence complementary to the first region of the corresponding target nucleic acid 10. The invading oligonucleotide 14 and the primary cleavage oligonucleotide 10 are arranged in a parallel orientation relative to one another.

The oligonucleotides of the invention are designed in accordance with methods that are known in the art. An initial consideration in choosing the length of oligonucleotides is the temperature under which they will be expected to be utilized for the methods described herein. For example, the chosen length of an oligonucleotide might vary depending on the thermal stability of the cleavage means. Longer oligonucleotides are generally expected to have a higher hybridization specificity. It is desirable that the oligonucleotides of the invention have a length that is long enough to be reasonably expected to hybridize only to the intended target sequence within a complex sample. The oligonucleotide probes of the invention are typically 10 to 40 nucleotides in length, and more typically 25 to 35 nucleotides in length. Where only a portion of a particular oligonucleotide is expected to anneal to a target nucleic acid strand, the length of the annealing portions are typically 10 to 25 nucleotides in length, and more typically 15 to 20 nucleotides in length. It is not intended that the method of the present invention be limited to any particular size of the primary cleavage oligonucleotide or invading oligonucleotide.

Another parameter that is selected is the degree to which the upstream invading oligonucleotide sequence overlaps into the downstream primary cleavage oligonucleotide sequence. This affects the sizes of the cleavage products resulting from cleavage of the primary cleavage oligonucleotide.

An important feature of the method is that the primary cleavage oligonucleotide can depart from the target nucleic acid after being cleaved, thus permitting the annealing and cleavage of other copies of the primary cleavage oligonucleotides (“turnover”) without any discrete denaturation or displacement steps. The ability of the primary cleavage oligonucleotides to turnover can be facilitated by the design of the primary cleavage oligonucleotides. The Tₘ of the primary cleavage oligonucleotide is a function of the full length of that primary cleavage oligonucleotide. Accordingly, the Tₘ of the primary cleavage oligonucleotide equals the Tₘ of the cleavage portion and the 3′, portion. The cleavage portion of the primary cleavage oligonucleotide is released upon cleavage, leaving the 3′ portion annealed to the target nucleic acid. If the Tₘ of 3′ end of the primary cleavage oligonucleotide is less than the reaction temperature, and the reaction temperature is less than the Tₘ of the entire primary cleavage oligonucleotide, then cleavage of the primary cleavage oligonucleotide will lead to the departure of 3′ end of the primary cleavage oligonucleotide and a new primary cleavage oligonucleotide will be able to hybridize. In an embodiment, the primary cleavage oligonucleotide is designed so that after cleavage of a portion of the primary cleavage oligonucleotide, the Tₘ of the remainder of the primary cleavage oligonucleotide is below the reaction temperature.

The primary cleavage oligonucleotide may hybridize inefficiently to the target nucleic acid if the binding of the 3′ portion of the invading oligonucleotide to the target nucleic acid is more stable than the binding of the cleavage portion of the primary cleavage oligonucleotide (for example, if the 5′end of the invading oligonucleotide is long or is rich in G-C basepairs), then the 3′end of the invading oligonucleotide may be favored in the competition for binding to the second region of the target. Alternatively, if the 3′ portion of primary cleavage oligonucleotide binds particularly strongly to the target nucleic acid, then the invading oligonucleotide will still cause internal cleavage of the primary cleavage oligonucleotide, but the 3′ portion of the primary cleavage oligonucleotide bound to the third region of the target nucleic acid may not dissociate at the reaction temperature. This is likely to reduce the detection signal because the turnover would be less than optimal. Thus, it is advantageous for the portions of the oligonucleotides that anneal with the first and third regions of the nucleic acid target have similar melting temperatures.

The one or more samples comprising target nucleic acid, and in preferred embodiments more than one target nucleic acid, primary cleavage oligonucleotide(s), and invading oligonucleotide(s) are assembled into a buffered reaction mixture suitable for nucleic acid modification enzymes, the components of which are well known to those of skill in the art. Typically, the primary cleavage oligonucleotides are provided in sufficient excess so that the rate of hybridization to the target nucleic acid(s) is rapid. The reactions are typically performed with 5 to 10 pmoles of each primary cleavage oligonucleotide, 1 pmol of each invading oligonucleotide, to 1 fmol of each target nucleic acid per reaction mixture. Other oligonucleotide concentrations, either higher or lower, commonly used in the art are contemplated and the methods described herein are not limited to these amounts.

It is advantageous for invading oligonucleotides to be immediately available to direct the cleavage of each primary cleavage oligonucleotide that hybridizes to a target nucleic acid. However, because the invading oligonucleotide is not cleaved and is reusable, the primary cleavage oligonucleotide is provided in excess of the invading oligonucleotide in the reaction. An exemplary ratio of primary cleavage oligonucleotide to invading oligonucleotide are between a 2 and a 100 fold excess of primary cleavage oligonucleotide over invading oligonucleotide. These ratios are not intended to limit the scope of the invention and other ratios may be employed.

Nucleic Acid Cleavage Means

A nucleic acid cleavage means encompasses any means that is capable of cleaving a cleavage structure, including but not limited to enzymes. Typically, the nucleic acid cleavage means comprise one or more “structure-specific nucleases”. These structure-specific nucleases recognize specific secondary structures in a nucleic acid molecule and cleave these structures.

The nucleic acid cleavage means includes native DNA polymerases having 5′ nuclease activity (e.g., Taq DNA polymerase, E. coli DNA polymerase I), nuclease activity provided from a variety of sources including the Cleavase enzymes, the FEN-1 endonucleases (including RAD2 and XPG proteins), Taq DNA polymerase and E. coli DNA polymerase I. In preferred embodiments the cleavage means includes DNA polymerases that have been modified so as to have a 5′ nuclease without synthetic activity. These 5′ nucleases are capable of cleaving naturally occurring structures in nucleic acid templates by structure-specific cleavages. The nucleic acid cleavage means further encompasses 5′ nucleases derived from thermostable Type A DNA polymerases that retain 5′ nuclease activity but have reduced or absent synthetic activity. Typically, a 5′ nuclease having reduced synthetic ability retains substantially the same 5′ exonuclease activity as the native DNA polymerase. This means that the modified enzyme retains the ability to function as a structure-dependent single-stranded endonuclease, but not necessarily at the same rate of cleavage as compared to the unmodified enzyme.

Modified enzymes having reduced synthetic activity and increased 5′ nuclease activity relative to unmodified enzymes are also envisioned by the present invention. Typically, the modified enzyme may have no synthetic activity remaining or may have that level of synthetic activity that will not interfere with the use of the modified enzyme in the detection assay described below. Exemplary nucleic acid cleavage means are known in the art, and are described in more detail in U.S. Pat. No. 5,994,069 to Hall et al., U.S. Pat. No. 5,719,028 to Dahlberg et al., U.S. Pat. No. 5,837,450 to Dahlberg et al., the contents of which are all hereby incorporated by reference in their entirety.

Primary Stage Cleavage

A “cleavage structure” refers to a structure that is formed by the interaction of an oligonucleotide and a target nucleic acid to form a duplex, where the resulting structure is cleavable by a cleavage means. The cleavage structure is a substrate for specific cleavage by the cleavage means, as opposed to a nucleic acid molecule that is a substrate for non-specific cleavage by agents such as phosphodiesterases that cleave nucleic acid molecules without regard to secondary structure (i.e., no formation of a duplexed structure is required). The products generated by the reaction of a cleavage means with a cleavage structure are referred to herein as “cleavage products”.

A primary cleavage structure is generated for each target nucleic acid by the interaction of each target nucleic acid with the corresponding primary cleavage oligonucleotide and the corresponding invading oligonucleotide. The primary cleavage reaction refers to that which occurs first, in response to the formation of the cleavage structure on the target nucleic acid. In each primary cleavage structure, at least the 3′ portion of each primary cleavage oligonucleotide is annealed to a corresponding target nucleic acid. As described above, the 3′ portion of the primary cleavage oligonucleotide anneals to the third region of the corresponding target nucleic acid. At least the 5′ portion of each invading oligonucleotide is annealed to a first region of the corresponding target nucleic acid.

The primary cleavage structures are cleaved by the nucleic acid cleavage means to generate a cleavage product termed herein as a “cleaved assay specific probe”. There is a unique cleaved assay specific probe formed corresponding to each target nucleic acid. Each cleaved assay specific probe comprises a 5′ region and a 3′ region.

Secondary Cleavage

The improvements on invading-based cleavage assays of the present invention provide an unexpected increase in multiplexing capacity that enables the simultaneous analysis of numerous target nucleic acids. These improvements are enhanced by performing a secondary cleavage reaction where the cleavage product of the first stage interacts with a unique secondary cleavage oligonucleotide for each target and forms a secondary cleavage structure. Subsequent sequential cleavage reactions are within the scope of the invention, including tertiary cleavage reactions and so forth. The cleavage of the secondary cleavage structure or formation of second stage cleavage products can be used as a basis of detection or to drive further stages of sequential cleavage reactions. Importantly, the product of the secondary cleavage reaction is not capable of initiating either of the first or second stage cleavage reactions.

In one reaction format, the reaction components for the first stage cleavage are mixed with components for the second stage cleavage reaction so that second stage reactions can be initiated directly after product from the primary cleavage reaction becomes available. In this format the primary and secondary cleavage events can take place simultaneously. The reaction format can also be configured with each step of cleavage reactions being spatially or temporally separated, such as performing each stage in different reaction vessels or by causing a change in reaction conditions that allow later cleavage events to take place.

As shown in FIG. 1, in a preferred embodiment, each secondary cleavage oligonucleotide comprises contiguous first and second regions. The first region 40 comprises the 3′ end of the secondary cleavage oligonucleotide. The second region 42 comprises the 5′ end of the secondary cleavage oligonucleotide. The second region of the secondary cleavage oligonucleotide further comprises a region of self-complementarity that is capable of forming a hairpin loop.

For each specific target nucleic acid, the first region of each secondary cleavage oligonucleotide is complementary to a corresponding cleaved assay specific probe that is generated in the primary cleavage reaction. Typically, a portion of the second region of each secondary cleavage oligonucleotide is complementary to at least a portion of the 3′ region of the corresponding cleaved assay specific probe.

The amount of secondary cleavage oligonucleotide in the reaction mixture is typically between 5 pmoles and 10 pmoles per reaction. Other oligonucleotide concentrations, either higher or lower, commonly used in the art are contemplated and the methods described herein are not limited to these amounts.

Detection

Each secondary cleavage oligonucleotide typically comprises a detectable label or moiety. The label can be placed at either the 5′ or 3′ end of the secondary cleavage oligonucleotide, or may be positioned anywhere along the oligonucleotide. The detectable label can be dye such as a fluorophore. In preferred embodiments, the oligonucleotides comprise a fluorescent donor and fluorescent acceptor that generate a fluorescence-based signal in response to a change in distance between the fluorescent donor and acceptor that is caused by cleavage. In preferred embodiments, the 5′ end of each secondary cleavage oligonucleotide comprises one or more detectible labels. In the preferred embodiment illustrated in FIG. 1, the 5′ end of each secondary cleavage oligonucleotide comprise a fluorescent donor and fluorescent acceptor that generate a fluorescence-based signal in response to cleavage of the secondary cleavage oligonucleotide.

Various methods of detecting nucleic acids by using combinations of a fluorophore and an interacting molecule or moiety are described in Morrison, L. E., Halder, T. C. and Stols, L. M., “Solution phase detection of polynucleotides using interacting fluorescent labels and competitive hybridization”, Analyt. Biochem. 183, 231-244 (1989); Morrison, L. E. and Stols, L. M., “Sensitive fluorescence-based thermodynamic and kinetic measurements of DNA hybridization in solution”, Biochemistry 32, 3095-3104 (1993); Gelfand et al. U.S. Pat. No. 5,210,015, and Livak et al. U.S. Pat. No. 5,538,848, the content of all of the preceding are hereby incorporated herein by reference in their entirety.

Detecting Multiple Target Nucleic Acids

The invention further provides an analytical assay system for detecting more than one target nucleic acid at a time. In preferred embodiments, each secondary cleavage oligonucleotide comprises a coded bead attached thereto. In the embodiment illustrated in FIG. 1, a distinct dye coded bead 24 is attached to the 3′ end of each secondary cleavage oligonucleotide. The analytical assay system and coded beads are described more completely in copending U.S. patent application Ser. No. 09/990,678, filed Nov. 14, 2001, by Michael L. Bell, et al., entitled “Analyte Detection System”; the contents of which are hereby incorporated by reference in their entirety. The method of the present invention utilizes fluorescent labeled beads to distinguish between numerous sub-populations of beads and to detect multiple target nucleic acids. The fluorescent labels employed can be excited by a common source and emit at distinguishable wavelengths from themselves and other fluorescent sources in the system. The fluorescent labels typically have excitation wavelengths in the red, far-red or near-infrared region of the spectrum.

According to an embodiment of the invention, multiple target nucleic acids are simultaneously detected and measured by combining fluidics or microfluidics and fluorescent bead sensor technology. Multiple analytical reactions are isolated onto a set of micrometer scale beads, which are read individually by a device such as a flow cytometer or static bead reader. The device determines the identity of each type of bead and the extent to which each bead has reacted with its corresponding analyte. Each set of beads: 1) carries a unique combination of fluorescent identification labels to code the beads; 2) is specific to an analyte, or class of analytes of interest; and 3) contains or is capable of binding to a fluorescent dye coupled oligonucleotide for identifying individual analytes of interest (i.e., an analytical dye, or a fluorescent analyte detection dye).

The beads employed in the present invention are generally made of polymeric materials such as a polystyrene. Suitable preparation techniques are generally known to those skilled in the art to make beads/particles that are useful in the present invention. An example of a suitable preparation technique is described in U.S. Pat. No. 4,609,689, incorporated herein by reference. Alternatively, the beads/particles may be obtained from a commercial supplier such as Bangs Laboratories Inc.

The fluorescent bead encoding labels employed in the invention are preferably, but not required to be, embedded or trapped within the bead. Internally embedding the fluorescent labels in the bead increases signal stability by shielding the labels from environmental factors that cause fluorescence degradation. Internally embedding the fluorescent labels in the bead also reserves the exterior of the bead for binding analytes and/or analytical dyes.

The fluorescent identification labels are added to the beads by using methods known to those in the art. One known method is a casting process, such as the casting process described in U.S. Pat. Nos. 4,302,166 and 4,162,282, which are incorporated herein by reference. In this process, a fluorescent label and a polymer are dissolved in a solvent. The solution is expelled as a stream through a fine nozzle into a sheath of water. A piezoelectric transducer breaks the stream up into discrete droplets that cure into beads as the solvent diffuses into the water. Another process is the swell-shrink method. This method, which is incorporated herein by reference, is described by L. B. Bangs (Uniform Latex Particles; Seragen Diagnostics Inc. 1984, p. 40). The swell-shrink process consists of adding an oil-soluble or hydrophobic dye to stirred beads and after an incubation period, any dye that has not been absorbed by the beads is washed away.

A set of beads is distinguishable from another set of beads on the basis of a unique combination of fluorescent labels for coding the beads. Multiple sets of beads can be used to specifically detect multiple analytes in a single reaction. Detecting multiple analytes in one reaction can simplify multiple assay procedures and result in less variability between results arising from separate assays.

In the present invention, differing amounts of fluorescent labels are used in varying combinations in different sets of beads to identify an individual set of beads from another set of beads. It is preferable, but not required, that the beads are labeled with at least two fluorescent labels and greater numbers of label combinations can be used to create greater numbers of bead populations. For example, a bead containing one part label A and two parts label B is distinguishable from a second bead containing two parts label A and one part label B. These beads are distinguishable from a third bead containing two parts label A and four parts label B or four parts label A and two parts label B. Pairs of fluorescent labels can be used in this manner to multiply the number of distinguishable bead populations. Accordingly, if an analytical detection system is capable of distinguishing ten different amounts of label A, then label A alone could be used to differentiate only ten different bead populations. However, if an analytical detection system can additionally distinguish between ten different amounts of label B, label A and label B can be used in combination to fluorescently label the identities of ten times ten, or one hundred different bead populations. If a third label is employed, the number of identifiable beads expands to one thousand distinguishable bead populations.

For an optimal number of distinct bead species it is advantageous that the emission spectra of the fluorescent bead labels accurately correspond to the concentrations of different fluorescent labels employed in particular bead sets. For accurate identification and quantification of multiple analytes on beads by fluorescence it is also advantageous that there is minimal interference between extraneous sources of fluorescence, the fluorescent labels employed in the assay, and the fluorescent dye associated with the analyte.

Bead size is another parameter for coding beads. Beads may be commercially purchased in preformed sizes or prepared in different homogenous sizes. Preferred, but not required sizes of beads are 3.4, 4.5, 5.5, 7.0, and 10.2 microns. The size of a bead can be separately detected and determined apart from fluorescence and correlated, along with the fluorescent labels, with the analyte detection dye to detect and quantify an analyte of interest. If fewer numbers of coded beads are needed, a combination of fluorescent labels to mark beads is preferred.

The concentration of the fluorescent labels in the beads is proportional to the magnitude of the emission signal. The maximum number of distinguishable bead combinations is achieved by preparing beads with the same magnitude of emission signals. It is desirable, but not required, that the emission signals of different sets of beads of different sizes are of the same approximate magnitude. To achieve this objective, the concentration of fluorescent labels in small beads is increased, and/or the concentration of fluorescent labels in large beads is decreased. The emission wavelengths of the fluorescent labels used in the invention are in the near-infrared region of the electromagnetic spectrum. For purposes of this disclosure, the red or near infrared region of the electromagnetic spectrum is light having a wavelength greater than 750 nm and less than 1000 nm. The absorbance and emission spectra of these fluorescent labels are well removed from the spectra of common interferents. The long emission wavelengths of the fluorescent labels employed in the present invention enable a large selection of sensing dyes to be employed as the analytical signal for detecting multiple analytes of interest. Accordingly, fluorescent dyes having emission wavelengths less than 750 nm can be included as candidates for analytical sensing dyes without consideration of overlapping emission spectra with the fluorescent labels.

It is desirable, but not required, that the fluorescent labels be stable, both in the solvents employed for preparing the coded beads and in the beads themselves during storage and use. This includes conditions of use wherein the beads are repeatedly heated almost to the boiling point of water. Also, it is desirable, but not required, that the fluorescent labels to be employed for coding beads are soluble in the solvents required for infusing them into the beads. The fluorescent labels advantageously do not leach out of the beads during extended storage in aqueous media, or during high temperature processes employed in various assays such as DNA amplification.

It is also desirable, but not required, that the fluorescent labels in a set do not significantly interact through energy transfer, even when embedded in a single bead. Such interactions can result in inaccurate fluorescence detection (e.g., an apparent loss of fluorescence of a shorter wavelength dye in the presence of a longer wavelength dye). These types of interactions may complicate simultaneous use of the dyes as bead labels. Further, the fluorescent labels advantageously do not have significant interference with fluorescent dyes used as the analytical dye such as ETH 5294, a fluorescent pH indicator in bead optodes for measurement of target cations.

It is advantageous, but not required, that the fluorescent labels share the same excitation laser. The detection system is generally more compact when the same excitation laser is employed in the system and the use of one laser to excite the fluorescent label combination is generally more economically efficient. However, multiple excitation lasers may be employed in the detection system to excite the fluorescent label combinations in alternate embodiments.

The emission wavelengths of the fluorescent labels, when used in combination in a bead, are generally distinguishable from one another, but can have overlapping portions. A distinguishable fluorescent label combination is such that one particular bead with one combination of fluorescent labels can be identified or differentiated from another bead with a different combination of fluorescent labels by the particular emission spectra of each bead. For example, a first bead can be identified by comparing the relative magnitude of the spectral emissions of the fluorescent labels in that bead. This bead can be distinguished from a second bead that has a different relative magnitude of spectral emissions for the fluorescent labels in that bead. Fluorescent label combinations employing fluorescent labels with spectral emission maxima that differ from one another by about at least a 30 nm Stokes shift are generally distinguishable. However, this is not a requirement of the present invention and the precise separation of the fluorescent label spectral emission maxima required to practice the invention can differ with each particular combination of labels and the spectral resolution. In one embodiment, the maximum wavelength of the emitted light of the analyte detection dye is different from the first and second maximum wavelengths of the emitted lights of the fluorescent labels by at least 80 nm, and the first and second excitation wavelengths differ by at least 80 nm and one of the excitation wavelengths about 635 nm or greater.

Employing the above described fluorescent labels in the assay system solves the limitations of prior fluorescence based detection systems in that: I) the emission signals of the beads do not significantly interact with each other; 2) the analyte emission signals do not significantly interact with the emission signals of the bead; and 3) the emission signals of the beads and the analytes do not significantly interact with extraneous sources of fluorescence. In addition to the advantages recited above, the use of long wavelength fluorescers as labels permits the use of inexpensive and compact diode lasers and economical photon detectors.

Red and near infrared fluorescent compounds are known to those skilled in the art and can be employed in the present invention as fluorescent labels for coding beads. Suitable fluorescent compounds are selected according to the above criteria by methods known to those skilled in the art and can be employed in the present invention. For example, Webb, J. P., et al., Eastman Organic Chemical Bulletin, (1974), Vol. 46, No. 3; Pierce, B. M., et al., IEEE Journal of Quantum Electronics, (July 1982), Vol. QE-18, No. 7, pp. 1164-1170; Strekowski, et al., J. Org. Chem., (1992), Vol. 57, pp. 4578-4580; and U.S. Pat. Nos., 2,887,479; 2,895,955; and 5,061,618, the disclosures of which are incorporated herein by reference, describe red and near infrared fluorescent compounds. In one embodiment cyanine dyes are used as fluorescent labels for coding the beads. The structures of these cyanine chromophores are described in detail in copending application Ser. No. 09/990,678 which is incorporated herein by reference. Dyes useful for coding beads include, for example, Phycoerythrin (620 nm emission), Fluorescein (518 nm emission), Squaraine (660 nm emission), DB CyS-C1 (715 emission), YL22 (860 nm emission), JM5488-72 (820 nm emission), Cy3 (570 nm emission), DB Cy3 (604 nm emission).

Fluorescent analyte detection dyes are known to those of skill in the art. The fluorescent analyte detection dye can be a single fluorescer or a donor-receptor dye pair that is activated by energy transfer in the detection system and can be synthetic or a naturally occurring fluorescer. Appropriate fluorescent analyte detection dyes can be selected for a particular assay and used in accordance with the present invention by those of skill in the art with reference to this disclosure.

The fluorescent analyte detection dyes are complexed to the bead by various methods known to those skilled in the art depending on the particular assay employed in a specific analytical reaction.

The coded beads allow multiple target nucleic acids to be detected simultaneously in an automated system. For example, a panel of beads may be prepared, composed of multiple subpopulations of beads, where each individual subpopulation of beads is specific to a different analyte of interest. The panel of beads is allowed to react with a test sample and then passed through the detection system. In this manner, a panel of analytes may be simultaneously detected and quantified. Thus, the invention is time efficient in that multiple assays may be completed in one reaction. Examples of panels known to those skilled in the art that may be used with the invention include electrolyte panels, hormone panels, and such. It is understood that other multi-analyte panels are known to those with skill in the art, and can be employed in the detection system of the present invention, with reference to this disclosure.

In an exemplary embodiment, 500,000 bead linked oligonucleotides may be prepared by the following method. In a tube, add 10 μl of 1 M Morpholino Ethane Sulfonic acid (MES) (pH 4.7), 500,000 washed red dyed beads and water are combined to give a total volume of 18 μl. Equilibrate the mixture at room temperature for at least 1 hour. Add 2 μl of the oligonucleotide to be coupled to the bead. The concentration of the oligonucleotide to be coupled should be at 100 pmoles/μl. Weigh out a 10 mg aliquot of 1-Ethyl-3-(3-Dimethylaminopropyl) Carbodiimide (EDC) into a 1.7 ml tubes. Just before use, add 75 μl of water to the tube of EDC and vortex for 10 seconds to dissolve. Add 1.7 μl of the dissolved EDC solution to the tube of beads to be coupled. Vortex for 10 seconds and sonicate for five minutes. Place at 75° C. for 15 minutes.

Repeat the addition of EDC solution, vortexing, sonication and heating steps two more times. After the coupling reaction, wash the beads. Add 0.4 ml PBS/0.02% Tween20, vortex for 10 seconds, sonicate for 5 minutes and centrifuge for 10 minutes at 14,000 rpm, and remove the supernatant. Add 0.4 ml 2×SSC/0.02% Tween20, vortex for 10 seconds, sonicate for 5 minutes, centrifuge for 10 minutes at 14,000 rpm, and remove the supernatant. Add 0.2 ml 2×SSC/0.02% Tween20, vortex for 10 seconds, sonicate for 5 minutes, centrifuge for 10 minutes at 14,000 rpm and remove the supernatant. Resuspend beads in 100 μl 2×SSC/0.02% Tween20, vortex for 10 seconds and sonicate for 5 minutes. Coupled beads may be stored at 4° C.

A preferred assay system employed in the present detection system and methods is a flow cytometer. Flow cytometry systems are known to those in the art. A preferred flow cytometer is a modified Coulter XL flow cytometer with a 635 nm or 785 nm laser replacing the standard argon ion laser. The flow cytometer operates in the conventional manner known as will be understood by those with skill in the art with reference to this disclosure.

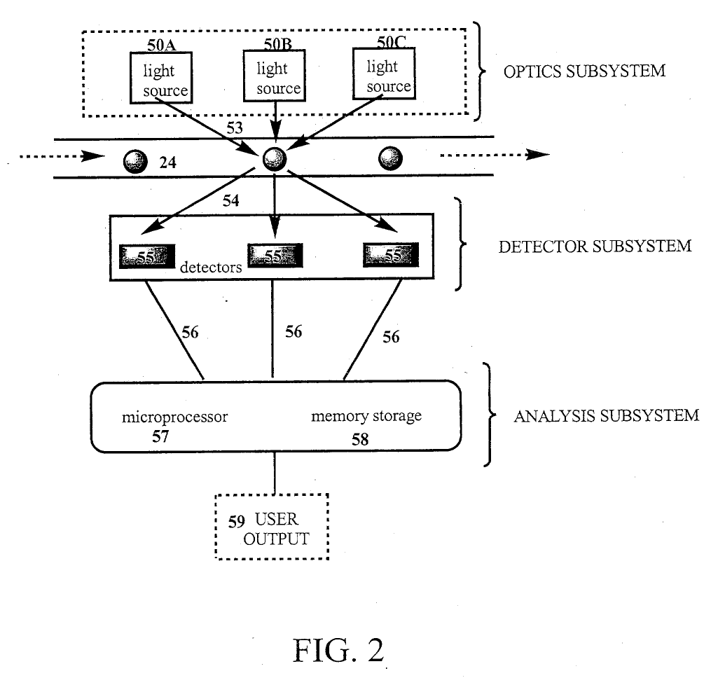

FIG. 2 is an exemplary schematic illustration of a flow cytometry system that can be used in the present invention. Light energy 53 is provided in the flow cytometer by exciting light sources 50A, 50B and 50C, such as a laser or an arc lamp, in the optics subsystem. Preferably, a longer wavelength excitation laser is used to simultaneously excite the fluorescent labels, used to mark the beads 24, and one or more shorter wavelength excitation lasers are used to excite the fluorescent analyte detection dyes. The optics subsystem of the cytometry device can include appropriate laser line filters, beam expanders, mirrors, lenses, and flowcells, as well as other components advantageous in operating a cytometry device as will be understood by those with skill in the art with reference to this disclosure.

Appropriate lower wavelength lasers for excitation of the analyte dyes are known to those skilled in the art. A preferred excitation wavelength for the fluorescent analyte detection dyes is a 532 nm diode laser, alternatively, a 635 nm diode laser, a 650 nm diode laser, or a 633 nm helium-neon laser can be used. Alternatively, a lower wavelength 488 nm argon-ion, or a 532 nm doubled YAG laser can be used. In another aspect of the invention, multiple detection lasers can be used to detect multiple fluorescent dyes at different excitation wavelengths. In this aspect of the invention, a combination of a higher wavelength laser with a lower wavelength laser is used. An example of this aspect of the invention is a 650 nm laser and a 532 nm laser, used to excite different fluorescent dyes on different beads. Longer wavelength lasers (e.g., greater than 750 nm) are known to those skilled in the art. A preferred laser excitation wavelength is about 635 nm or 785 nm. In a preferred, but not required aspect of the invention, a flow cytometry system with three lasers at 532, 650, and 780 nm is used.

Appropriate detectors 55 for detecting a particular emitting light 54 in the detection subsystem are known as will be understood by those with skill in the art with reference to this disclosure. The detectors can be photodiodes or photomultipliers or similar devices that convert light signals into electrical impulses thereby associating the detected light with its fluorescent source. Detectors for detecting forward and side scattered light are known to those in the art and can be used to detect light scatter in the detection system as will be understood by those with skill in the art with reference to this disclosure. Light scatter and fluorescence can be simultaneously detected with respect to each bead in the examination zone. In a preferred, but not required, aspect of the invention, a forward scatter detector, a side scatter detector, and photomultiplier tubes are employed in a detection subsystem. The detection subsystem can also employ a system of filters, mirrors, as well as other components advantageous in operating a cytometry device as will be understood by those with skill in the art with reference to this disclosure. The electrical signals from the detectors 56 are typically fed into the electronics of the system for signal and display processing, storage, and/or further processing.

In an analysis subsystem, hardware, such as a microprocessor 57 in combination with memory storage 58 such as a hard drive in a computer, collects detected data and processes the data. Suitable hardware used in the analysis system is known as will be understood by those with skill in the art with reference to this disclosure. The analysis system software, used for data and signal processing, can correlate detected data with known data to produce analytical results. The analysis subsystem can collect data from the electrical signals associated with each bead. A class of beads is established based on the common characteristics of the class of beads. The data from a known class of beads can be compared to the data detected from sample beads of an unknown class. The processed data and interpreted results can be given as output 59 to a user.

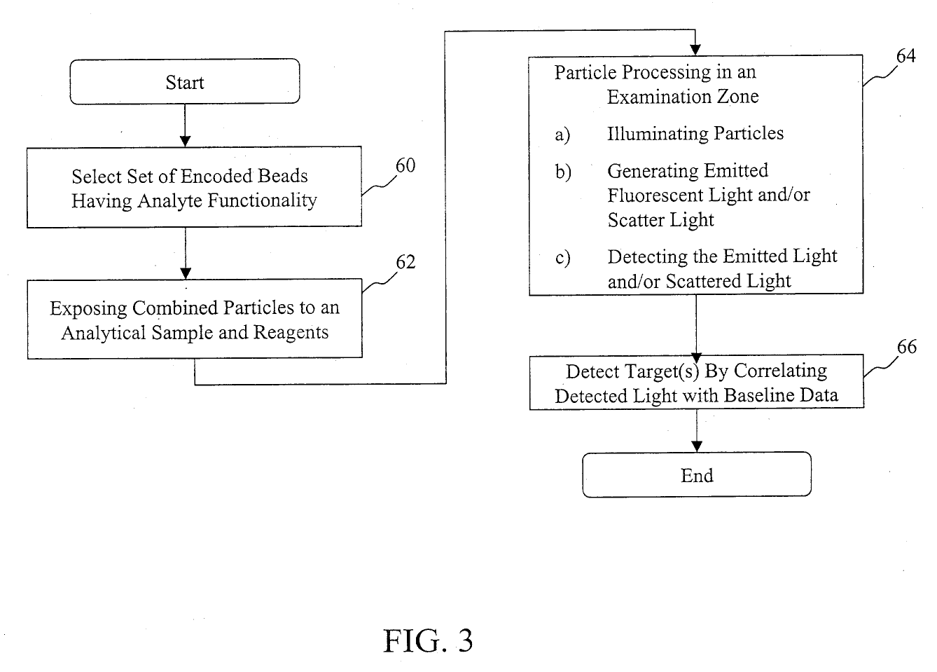

A method of using coded beads to detect target oligonucleotides is shown in FIG. 3. Encoded beads having analyte specific functionality are selected, box 60. The combined beads are then exposed to the sample and necessary reagents, box 62. Following exposure, the beads are processed in an examination zone. The beads are processed by illuminating the beads, generating emitted fluorescent light and/or scatter light and detecting the emitted light and/or scattered light, box 64. The detected emitted and/or scatter light data is correlated with the baseline data to determine the detection of one or more target nucleic acids, box 66.

According to the method of the present invention, an analytical sample is allowed to react with a set of beads specific to various analytes of interest. The beads are then passed through a detection device such as a flow cytometer. Beads that have reacted with their specific analyte of interest generate fluorescent emission spectra corresponding to the fluorescent dye associated with the particular bead and analyte of interest. The device identifies the beads at least partly by a unique combination of fluorescent labels incorporated into the beads. The information from the fluorescent labels is correlated with the information from the analyte specific fluorescent dye and the corresponding results allow quantitative identification of multiple analytes in one reaction.

### EXAMPLE 1

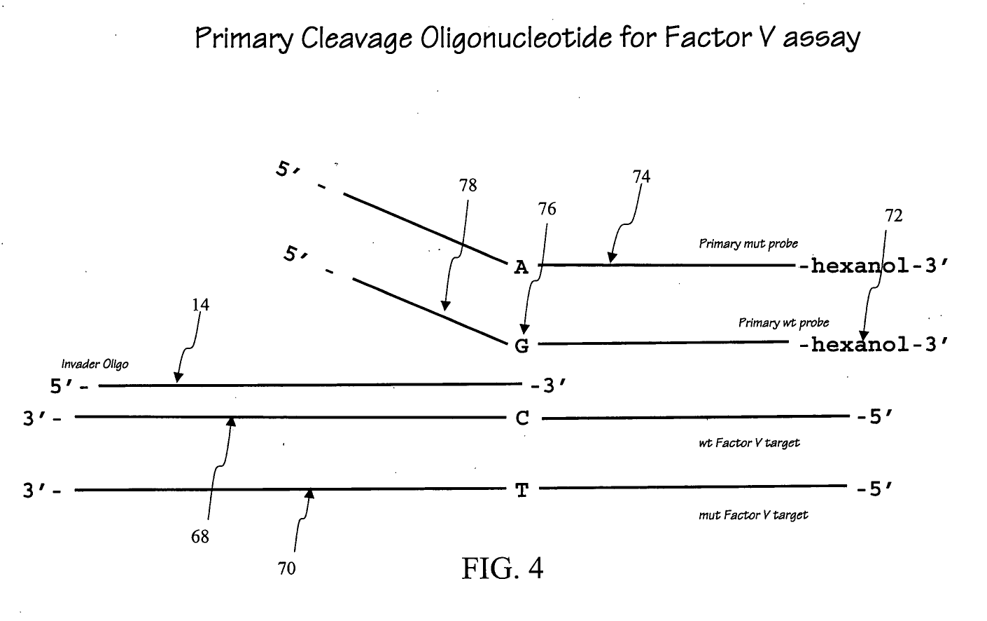

An experiment to detect the presence of wild type Factor V target nucleic acid from mutant Factor V target nucleic acid was conducted according to the preferred embodiment of the present invention. FIG. 4 shows a wild type Factor V target nucleic acid 68 and a mutant Factor V target nucleic acid 70. An invading oligonucleotide 14 specific to the Factor V target nucleic acid 68 is hybridized to the wild type Factor V target nucleic acid 68. Likewise, a primary cleavage oligonucleotide 72 specific for wild type target oligonucleotide is hybridized to the wild type Factor V target nucleic acid 68. Also visible is a primary cleavage oligonucleotide 74 specific for mutant Factor V target which has not yet hybridized to the wild type Factor V target oligonucleotide 70.

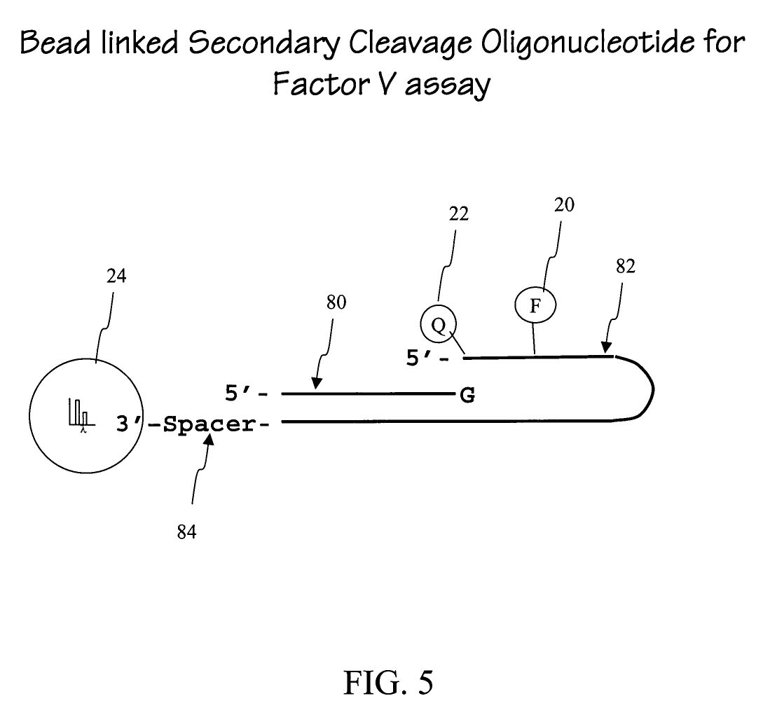

The hybridization of the invading oligonucleotide 14 leads to cleavage of the primary cleavage oligonucleotide 72 at a cleavage site 76 and the upstream cleavage portion 78 beyond the cleavage site is released as a cleaved assay specific probe 80. FIG. 5 shows a secondary cleavage oligonucleotide 82 coupled to a dye-coded bead 24, a fluor 20, and a quencher 22. The cleaved assay specific probe 80 hybridizes to the secondary cleavage oligonucleotide 82, and cleaves off the quencher. In an embodiment, a spacer 84 is inserted between the secondary cleavage oligonucleotide 82 and the dye-coded bead 24 to improve hybridization of the cleaved assay specific probe 80.

In the experiment, 10 amoles of Factor V target, both wild type and mutant, were combined with 1 pmole of invading oligonucleotide and 2 pmoles of wild type primary cleavage oligonucleotide. Also combined was 3 μl of Bead linked secondary cleavage oligonucleotide and 300 ng Cleavase VIII. The Factor V target, invading oligonucleotide, primary cleavage oligonucleotides and the Cleavase VIII were obtained from Third Wave Technologies, 502 South Rosa Road, Madison, Wis. 53719. All of the above ingredients were combined in a small tube in a total reaction volume of 20 μl and the reaction was carried out at 58 degrees Celsius for four hours.

Following the reaction, 150 μl of 2×SSC (0.3 M sodium chloride, 0.03 M sodium citrate buffer (a standard hybridization buffer), and 0.02% Tween 20 (a nonionic detergent) were added and the mixture was sonicated for 5 minutes. After sonication, the sample was analyzed on an Elite flow cytometer.

Although the desired result of example 1 was to only detect the wild type target, both wild type Factor V target nucleic acid and mutant Factor V target nucleic acid were separately identified. This was most likely because the reaction was not optimized and therefore the signal from the mutant target was much higher than desirable. A much greater amount of wild type Factor V target was detected.

Additional Embodiments

Several alternative embodiments of the present invention will now be described. The additional embodiments are meant to be illustrative, not restrictive.

First Additional Embodiment

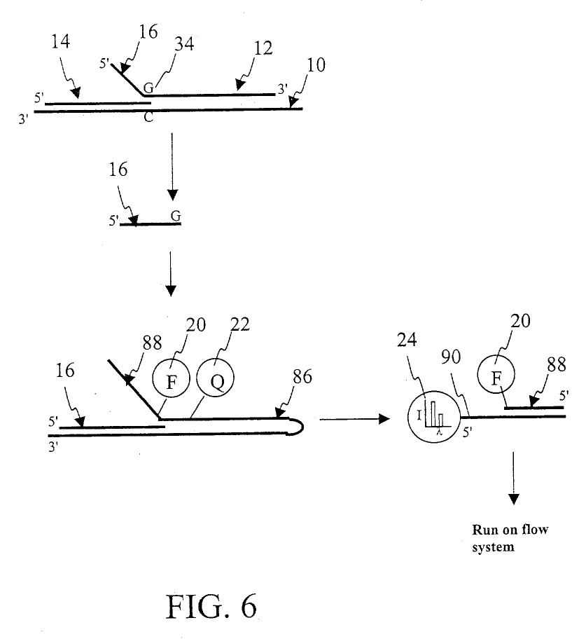

In a first additional embodiment, shown in FIG. 6, a target nucleic acid 10, is hybridized to a primary cleavage oligonucleotide 12. An invading oligonucleotide 14 also hybridizes to the target nucleic acid 10 to form a primary cleavage structure. A structure-specific nuclease cleaves the primary cleavage oligonucleotide 12 at a cleavage site 34, releasing a cleavage portion of the primary cleavage oligonucleotide 12 as a cleaved assay specific probe 16. The cleaved assay specific probe 16 hybridizes to a secondary cleavage oligonucleotide 86 having a fluor 20, and a quencher 22, leading to cleavage of a secondary assay specific probe 88 including the fluor 20 from the secondary cleavage oligonucleotide 86. The secondary assay specific probe 88 then hybridizes to a capture oligonucleotide 90 coupled to a dye encoded bead 24. In another embodiment, the orientation of the secondary assay specific probe and the capture oligonucleotide is designed so that the fluor is positioned away from the dye encoded bead.

### EXAMPLE 2

An experiment to detect the presence of wild type Factor V target nucleic acid from mutant Factor V target nucleic acid was conducted according to the first alternative embodiment of the present invention. Several different quantities (100, 10, 1, 0.1, 0.01 or 0.001 amoles) of Factor V target, both wild type and mutant, were combined with 10 pmoles of primary cleavage oligonucleotide, 1 pmole of invading oligonucleotide, 10 pmoles of secondary cleavage oligonucleotide, and 300 ng of Cleavase VIII. The primary cleavage oligonucleotide was a mixture of primary cleavage oligonucleotides specific to the wild type Factor V target and primary cleavage oligonucleotides specific to the mutant Factor V target. Likewise, the secondary cleavage oligonucleotide was a mixture of secondary cleavage oligonucleotide specific to the wild type Factor V target and secondary cleavage oligonucleotide specific to the mutant Factor V target. The Factor V target, invading oligonucleotide, primary cleavage oligonucleotide, secondary cleavage oligonucleotide and the Cleavase VIII were obtained from Third Wave Technologies, 502 South Rosa Road, Madison, Wis. 53719. The above ingredients were combined to give a total reaction volume 7 μl. The reaction mixture was kept at 63° C. for 4 hours.

Following the initial reaction, a capture hybridization reaction was carried out in 750 mM NaCl. 1 μl of bead linked capture oligonucleotide (1,000 or 2,000 beads) was added to the reaction mixture to give a total reaction volume of 10 μl. The bead linked capture oligonucleotide was a mixture of bead linked capture oligonucleotides specific for the wild type Factor V target and bead linked capture oligonucleotides specific for the mutant type Factor V target. The hybridization reaction involved a series of stepwise temperature reductions from 94 to 30° C. over 28° C. minutes.

Following the reaction, 150 μl of 2×SSC (0.3 M sodium chloride, 0.03 M sodium citrate buffer (a standard hybridization buffer), and 0.02% Tween 20 (a nonionic detergent) were added and the mixture was sonicated for 5 minutes. After sonication, the sample was analyzed on an Elite flow cytometer. Both wild type Factor V target nucleic acid and mutant Factor V target nucleic acid were separately identified, but a greater amount of wild type Factor V target was detected.

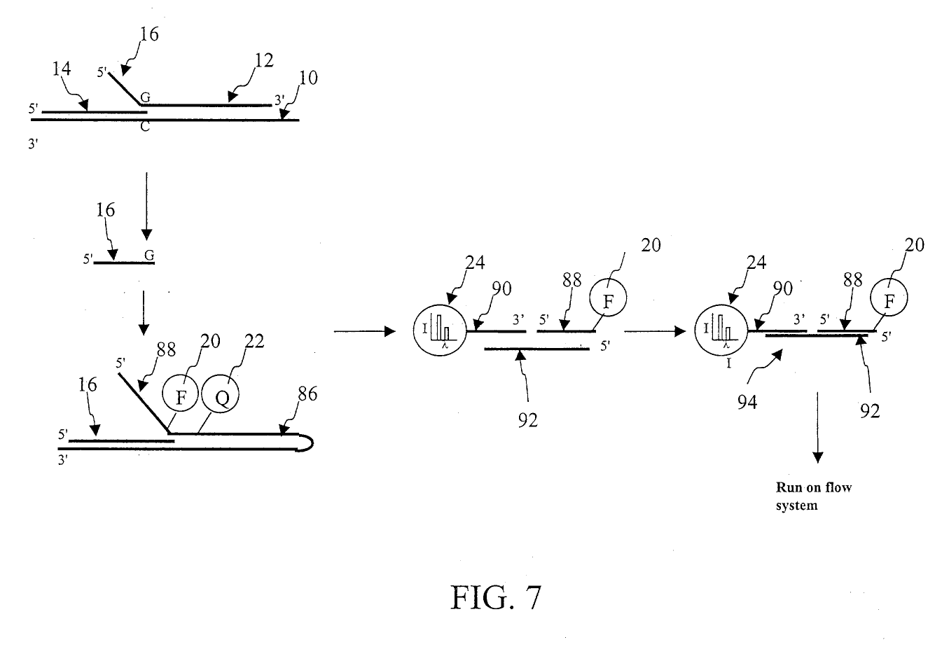

Alternatively, as shown in FIG. 7, a target nucleic acid 10 is hybridized to a primary cleavage oligonucleotide 12. An invading oligonucleotide 14 also hybridizes to the target nucleic acid 10 to form a primary cleavage structure. A structure-specific nuclease cleaves the primary cleavage oligonucleotide 12, releasing a cleaved assay specific probe 16. The cleaved assay specific probe 16 hybridizes to a secondary cleavage oligonucleotide 86 having a fluor 20, and a quencher 22, leading to cleavage of a portion of the secondary cleavage oligonucleotide 86, including the fluor 20, as a secondary assay specific probe 88. Both the secondary assay specific probe 88 and a reporting oligonucleotide 90 coupled to a dye encoded bead 24 are hybridized to a hybridization template oligonucleotide 92 to form a target-specific detection complex 94.

Second Additional Embodiment

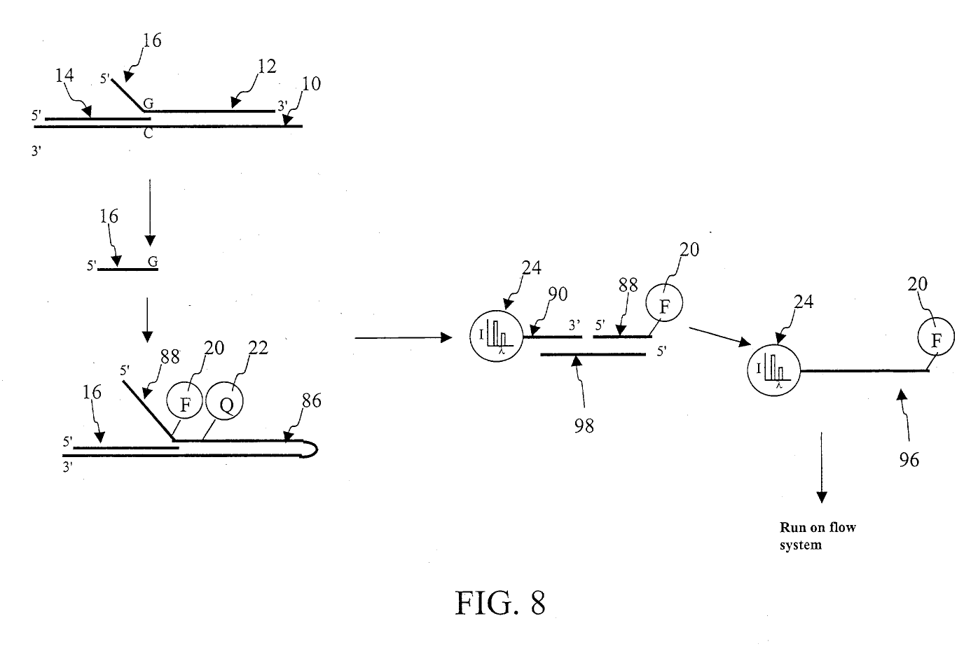

In a second additional embodiment, shown in FIG. 8, a target nucleic acid 10, is hybridized to a primary cleavage oligonucleotide 12. An invading oligonucleotide 14 also hybridizes to the target nucleic acid 10 to form a primary cleavage structure. A structure-specific nuclease cleaves the primary cleavage oligonucleotide 12, releasing a cleaved assay specific probe 16. The cleaved assay specific probe 16 hybridizes to a secondary cleavage oligonucleotide 86 having a fluor 20, and a quencher 22, leading to cleavage of a portion of the secondary cleavage oligonucleotide, including the fluor 20, as a secondary assay specific probe 88. The secondary assay specific probe 88 is then ligated to a reporter oligonucleotide 90 coupled to a dye encoded bead 24 to form a target-specific detection complex 96. The ligation of the secondary assay specific probe 88 and the reporter oligonucleotide 90 is facilitated by a template oligonucleotide 98 in conjunction with a DNA ligase (not shown).

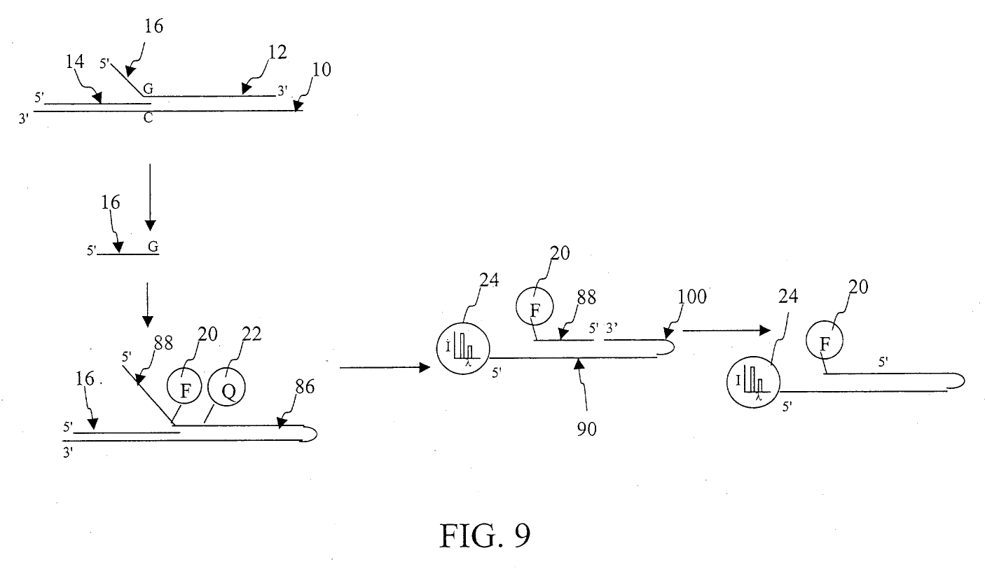

Optionally, as shown in FIG. 9, the reporter oligonucleotide 90 contains a hairpin loop 100. The hairpin loop 100 structure of the capture oligonucleotide eliminates the need for a separate template oligonucleotide.

### EXAMPLE 3

An experiment to detect the presence of wild type Factor V target nucleic acid from mutant Factor V target nucleic acid was conducted according to the second additional embodiment of the present invention. In the experiment, 10 amoles of Factor V target, either wild type or mutant, were combined with 1 pmole of invading oligonucleotide, 10 pmoles of wild type primary cleavage oligonucleotide, 10 pmoles of secondary cleavage oligonucleotide and 300 ng Cleavase VIII. The Factor V target, invading oligonucleotide, primary cleavage oligonucleotide, secondary cleavage oligonucleotide and the Cleavase VIII were obtained from Third Wave Technologies, 502 South Rosa Road, Madison, Wis. 53719. All of the above ingredients were combined in a small tube with a total reaction volume of 7 μl and the reaction carried out at 63 degrees Celsius for four hours.

Following the initial reaction, a ligation reaction was conducted in 1× Ligase buffer (20 mM Tris-HCl, pH 7.6, 25 mM potassium acetate, 10 mM magnesium acetate, 10 mM DTT, 1 mM NAD and 0.1% TritonX100). 1 μl of a 1× Ligase buffer, 40 units of Taq DNA Ligase, 1 μl of bead linked capture oligonucleotide containing about 2000 beads and 7 μl of the initial reaction mixture to give a total volume of 10 μl. The ligation reaction was conducted at 63 degrees Celsius for 1 hour.

Following the ligation reaction 150 μl of 1×PBS (phosphate buffered saline: 80 g NaCl, 2 g KCl, 11.5 g Na2HPO4.7H2O per liter) was added. The mixture was sonicated for 5 min and then analyzed on an Elite flow cytometer. This experiment was performed alongside a capture hybridization reaction and target was detected in both instances. It was unclear if ligation presented significant improvement over hybridization in this experiment, but ligation of a secondary assay specific probe to a bead linked capture oligonucleotide is believed to present an effective way of detecting the presence of a specific target oligonucleotide.

Third Additional Embodiment

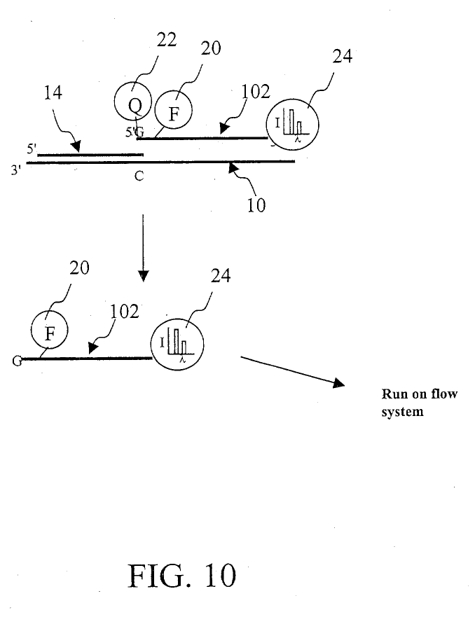

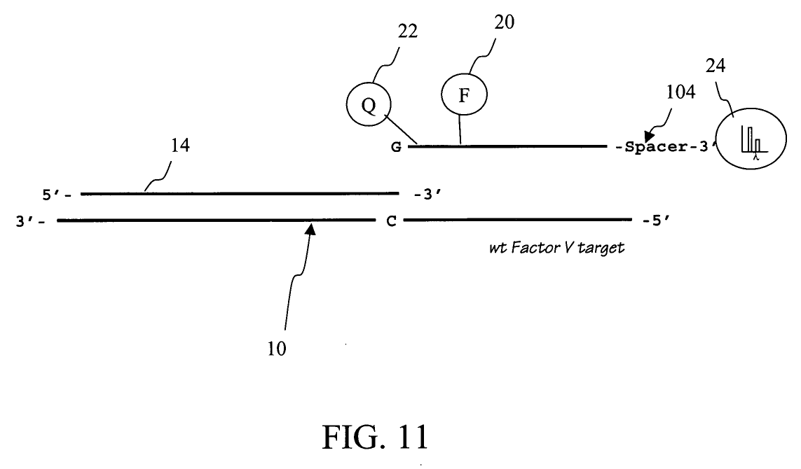

In a third additional embodiment of the present invention, the primary cleavage oligonucleotide is coupled to a dye encoded bead. As shown in FIGS. 10 and 11, the 3′ end of the primary cleavage oligonucleotide 102 is coupled to a dye-coded bead 24. The primary cleavage oligonucleotide 102 has a fluor 20 and a quencher 22. The invading oligonucleotide 14 allows cleaving of the quencher 22 from the primary cleavage oligonucleotide 102 when the primary cleavage oligonucleotide 102 is coupled to the target nucleic acid 10. In the presence of the proper target nucleic acid, the number of cleaved primary cleavage oligonucleotides increases linearly with increases in the incubation time.

In an embodiment, the primary cleavage oligonucleotide has a spacer 104 inserted between the upstream end and the dye-coded bead 24 to help hybridization of the invading and the target to the primary cleavage oligonucleotide. In another embodiment, the primary cleavage oligonucleotide has a flap extending at the 5′ position of the quencher.

### EXAMPLE 4

An experiment to detect the presence of wild type Factor V target nucleic acid from mutant Factor V target nucleic acid was conducted according to the third additional embodiment of the present invention. In the experiment, either 10, 100 or 1000 fmoles of mutant Factor V target was combined with 5 pmoles of invading oligonucleotide, 0.1 μl Bead linked primary cleavage oligonucleotide, 1 μg yeast tRNA and 600 ng of Cleavase VIII. The total reaction volume was 20 μl and the reaction was carried out at 58 degrees Celsius for 4 hours.

Following the reaction, 150 μl of sodium chloride, sodium citrate buffer (a standard hybridization buffer), and 0.02% Tween 20 (a nonionic detergent) were added and the mixture was sonicated for 5 minutes. After sonication, the sample was analyzed on an Elite flow cytometer. A significant amount of target was detected.

Fourth Additional Embodiment

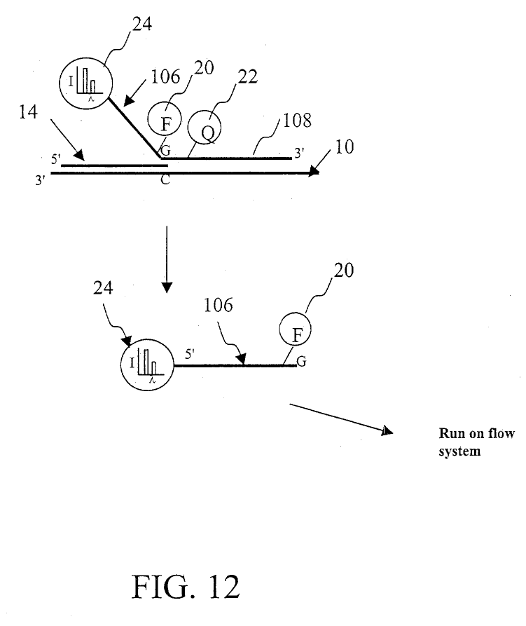

In a fourth additional embodiment, shown in FIG. 12, a dye coded bead 24 is attached to the 5′ end of a cleavage portion 106 of the primary cleavage oligounucleotide 108. The cleavage portion of the primary cleavage oligonucleotide 108 is also coupled to a fluor 20 and a quencher 22. Upon hybridization of the primary cleavage oligonucleotide 108 and an invading oligonucleotide 14 to a target oligonucleotide 10 and subsequent cleavage, the cleavage portion 106, including both the dye coded bead 24 and the fluor 20, is cleaved from the remainder of the primary cleavage oligonucleotide 108.

Fifth Additional Embodiment

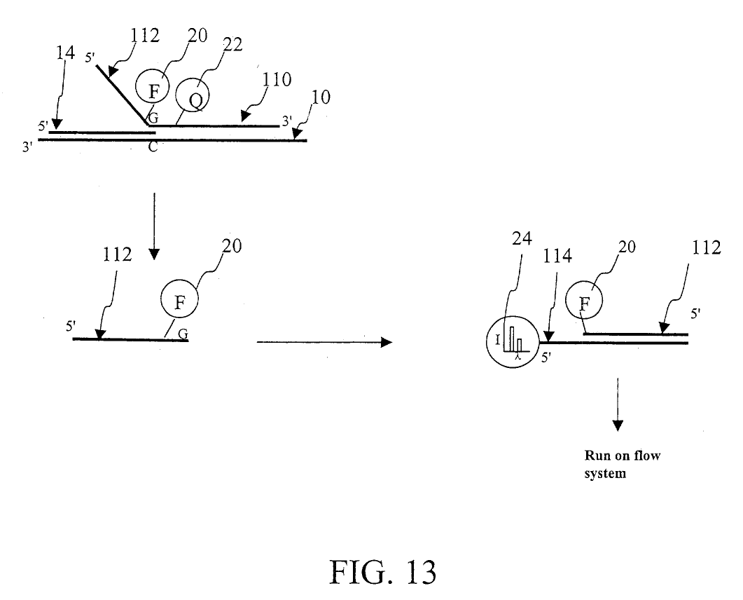

In a fifth additional embodiment, as shown in FIG. 13, a primary cleavage oligonucleotide 110 has a cleavage portion 112 with a fluor 20 and a quencher 22. Upon hybridization of the primary cleavage oligonucleotide 110 and an invading oligonucleotide 14 to a target oligonucleotide 10, the cleavage portion 112 with the fluor 20 is cleaved to form a primary assay specific probe 112. The primary assay specific probe 112 coupled to the fluor 20 is then hybridized directly to a capture oligonucleotide 114 coupled to a dye encoded bead 24.

Optionally, the orientation of the cleavage portion and the fluor is designed so that the fluor is positioned away from the dye encoded bead when the primary assay specific probe is hybridized to the capture oligonucleotide coupled to the dye encoded bead.

### EXAMPLE 5

An experiment to detect the presence of wild type Factor V target nucleic acid from mutant Factor V target nucleic acid was conducted according to the fifth additional embodiment of the present invention. In the experiment, different quantities (500, 100, 10, 1, 0.1 and 0.01 fmoles) of Factor V target (both wild type and mutant) were combined with 1 pmole Invading oligonucleotide, 10 pmoles primary probe (wild type and mutant) and 300 ng of Cleavase VIII, all of which were supplied by Third Wave Technologies, 502 South Rosa Road, Madison, Wis. 53719. The total reaction volume was 5 μl. The reaction temperature was 58° C. and the reaction time was 4 hours.

Following the initial reaction, the bead linked capture oligonucleotide was a mixture of bead linked capture oligonucleotides specific for the wild type Factor V target and bead linked capture oligonucleotides specific for the mutant type Factor V target.

Following the initial reaction, a capture hybridization reaction was carried out in 750 mM NaCl. 1 μl of the initial reaction volume was removed and added to 21 μl containing a mix of bead linked capture oligonucleotides for detecting wild type and mutant target (approx. 2,500 each) to give a reaction volume of 20 μl. The hybridization reaction involved a series of stepwise temperature reductions from 94° C. to 30° C. over 28 minutes.

Following the hybridization reaction, 250 μl of 2×SSC (a standard hybridization buffer), and 0.02% Tween 20 (A nonionic detergent) were added, and the mixture was sonicated for 5 minutes. After sonication, the sample was analyzed on an Elite flow cytometer. The amount of target detected increased with the amount of target present.

Having thus described the invention, it should be apparent that numerous modifications and adaptations may be resorted to without departing from the scope and fair meaning of the instant invention as set forth hereinabove and as described hereinbelow by the claims. Although the present invention has been described in considerable detail with reference to certain preferred versions thereof, other versions are possible. Therefore, the spirit and scope of the appended claims should not be limited to the description of the preferred versions described herein.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means” for performing a specified function or “step” for performing a specified function, should not be interpreted as a “means” or “step” clause as specified in 35 U.S.C. § 112.

What is claimed is:

1. A method for detecting the presence of a target nucleic acid molecule comprising:
a. providing:
i. a sample comprising at least one target nucleic acid, each target nucleic acid having a first region, a second region and a third region, wherein the first region is contiguous to and downstream from the second region and wherein the second region is contiguous to and downstream from the third region;
ii. a primary cleavage oligonucleotide corresponding to each target nucleic acid, each primary cleavage oligonucleotide having a 3′ portion and a cleavage portion, wherein the 3′ portion of each primary cleavage oligonucleotide comprises a sequence complementary to the third region of each corresponding target nucleic acid and the cleavage portion of each primary cleavage oligonucleotide comprises a sequence complementary to the second region of each corresponding target nucleic acid;
iii. an invading oligonucleotide corresponding to each target nucleic acid, each invading oligonucleotide having a 3′ portion and a 5′ portion, wherein the 3′ portion of each invading oligonucleotide comprises a sequence complementary to the second region of each corresponding target nucleic acid and the 5′ portion of each invading oligonucleotide comprises sequence complementary to the first region of each corresponding target nucleic acid; and
iv. a nucleic acid cleavage means;
b. generating a primary cleavage structure for each target nucleic acid, wherein at least the 3′ portion of the primary cleavage oligonucleotide is annealed to a corresponding target nucleic acid and at least the 5′ portion of the invading oligonucleotide is annealed to a corresponding target nucleic acid, wherein cleavage of the primary cleavage structure is performed by the nucleic acid cleavage means to cleave each primary cleavage oligonucleotide to generate a cleaved assay specific probe corresponding to each target nucleic acid, each cleaved assay specific probe having a 5′ region and a 3′ region;
c. providing a secondary cleavage oligonucleotide for each target nucleic acid, each secondary cleavage oligonucleotide having contiguous first and second regions, wherein the first region comprises a 3′ end of the secondary cleavage oligonucleotide, the 3′ end of the secondary cleavage oligonucleotide is coupled to a solid support, wherein the second region comprises a 5′ end of the secondary cleavage oligonucleotide, a region of self-complementarity, and is coupled to a detectible label in a first state, wherein the first region of each secondary cleavage oligonucleotide is complementary to a cleaved assay specific probe for a corresponding target nucleic acid, and wherein a portion of the second region of each secondary cleavage oligonucleotide is complementary to at least a portion of the 3′ region of the corresponding cleaved assay specific probe;
d. generating a secondary cleavage structure, wherein the 5′ end of each secondary cleavage oligonucleotide is cleaved from the remainder of the second region of each secondary cleavage oligonucleotide changing the detectible label to a second state; and
e. detecting the cleavage of the secondary cleavage oligonucleotides by a change in fluorescence, thereby detecting the target nucleic acid corresponding to the secondary cleavage oligonucleotide.

2. The method of claim 1, wherein the solid support coupled to the 3′ end of each secondary cleavage oligonucleotide comprises a coded bead.

3. The method of claim 2, wherein:
a. the detectible label coupled to the secondary cleavage oligonucleotide is a fluorescent analyte detection dye, the analyte detection dye being excitable by light at a first excitation wavelength and capable of emitting light at a maximum wavelength when excited; and
b. beach coded bead has an identification label comprising at least one fluorescent label, the fluorescent identification label being excitable by light of a second excitation wavelength and capable of emitting light at a maximum wavelength, wherein the maximum wavelength of the emitted light of the fluorescent analyte detection dye is different from the maximum wavelength of the emitted light of the fluorescent identification label by at least 80 nm, the first and second excitation wavelengths differ by at least 80 nm, and one of the excitation wavelengths is about 635 nm or greater.

4. The method of claim 3 wherein the identification label comprises at least two fluorescent labels in a combination of relative amounts, the fluorescent identification labels being excitable by light of a same second excitation wavelength and capable of emitting lights at maximum wavelengths, distinguishable from each other.

5. The method of claim 3, wherein the fluorescent identification labels are embedded within the bead.

6. The method of claim 3, wherein light at the first excitation wavelength causes substantially no emitted light by the at least one fluorescent identification label and light at the second excitation wavelength causes substantially no emitted light by the analyte detection dye.

7. The method of claim 3 wherein different fluorescent analyte detection dyes are coupled to secondary cleavage oligonucleotides corresponding to different target nucleic acids.

8. The method of claim 3 wherein the first excitation wavelength is less than about 635 nm and the second excitation wavelength is about 635 nm or greater.

9. The method of claim 3, wherein the fluorescent analyte detection dye comprises a fluorescent donor and a fluorescent acceptor that function as a fluorescent donor/acceptor pair capable of fluorescence resonance energy transfer with each other in response to activation of the fluorescent donor by light of a predetermined wavelength or band of wavelengths.

10. The method of claim 3, wherein the fluorescent analyte detection dye comprises a fluorescent reporter and a quencher molecule, the quencher molecule being removed by cleavage of the secondary cleavage oligonucleotide.

11. The method of claim 1, wherein the solid support attached to the 3′ end of each secondary cleavage oligonucleotide comprises a surface and each secondary cleavage oligonucleotide is immobilized to a distinct and separate location on the surface.

12. The method of claim 1 1, wherein the one or more detectable labels comprise a fluorescent donor and a fluorescent acceptor that function as a fluorescent donor/acceptor pair capable of fluorescence resonance energy transfer with each other in response to activation of the fluorescent donor by light of a predetermined wavelength or band of wavelengths.

13. The method of claim 1, wherein the method is used to detect single nucleotide polymorphisms.

14. The method of claim 1, wherein the nucleic acid cleavage means comprises a structure-specific nuclease.

15. The method of claim 1, wherein the nucleic acid cleavage means comprises a thermostable 5′ nuclease.

16. The method of claim 1, wherein the target nucleic acid comprises single-stranded DNA.

17. The method of claim 1, wherein the target nucleic acid comprises double-stranded DNA.

18. The method of claim 1, wherein the target nucleic acid comprises RNA.

19. A method of detecting the presence of a target nucleic acid comprising:
a. providing:
i. a sample comprising at least one target nucleic acid, each target nucleic acid having a first region, a second region and a third region, wherein the first region is contiguous to and downstream from the second region and wherein the second region is contiguous to and downstream from the third region;
ii. a primary cleavage oligonucleotide corresponding to each target nucleic acid, each primary cleavage oligonucleotide having a 3′ portion and a cleavage portion, wherein the 3′ portion of each primary cleavage oligonucleotide comprises a sequence complementary to the third region of each corresponding target nucleic acid and the cleavage portion of each primary cleavage oligonucleotide comprises a sequence complementary to the second region of each corresponding target nucleic acid;
iii. an invading oligonucleotide corresponding to each target nucleic acid, each invading oligonucleotide having a 3′ portion and a 5′ portion, wherein the 3′ portion of each invading oligonucleotide comprises a sequence complementary to the second region of each corresponding target nucleic acid and the 5′ portion of each invading oligonucleotide comprises sequence complementary to the first region of each corresponding target nucleic acid; and
iv. a nucleic acid cleavage means;
b. generating a primary cleavage structure for each target nucleic acid, wherein at least the 3′ portion of the primary cleavage oligonucleotide is annealed to a corresponding target nucleic acid and at least the 5′ portion of the invading oligonucleotide is annealed to a corresponding target nucleic acid, wherein cleavage of the primary cleavage structures are performed by the nucleic acid cleavage means to cleave each primary cleavage oligonucleotide to generate a cleaved assay specific probe corresponding to each target nucleic acid, each cleaved assay specific probe having a 5′ region and a 3′ region;
c. providing a secondary cleavage oligonucleotide for each target nucleic acid, each secondary cleavage oligonucleotide having contiguous first and second regions, wherein the first region comprises the 3′ end of the secondary cleavage oligonucleotide, wherein the second region comprises a region of self-complementarity and a cleavage portion, the cleavage portion being coupled to a detectible label, wherein the first region of each secondary cleavage oligonucleotide is complementary to a cleaved assay specific probe for a corresponding target nucleic acid, and wherein a portion of the second region of each secondary cleavage oligonucleotide is complementary to at least a portion of the 3′ region of the corresponding cleaved assay specific probe;
d. generating a secondary cleavage structure, wherein the cleavage portion of each secondary cleavage oligonucleotide is cleaved from the remainder of the second region of each secondary cleavage oligonucleotide;
e. providing a capture oligonucleotide for each secondary cleavage oligonucleotide comprising a sequence complementary to the cleavage portion of the secondary cleavage oligonucleotide, the capture oligonucleotide being coupled to a dye coded bead; and
f. detecting the cleavage products of secondary cleavage oligonucleotides by detecting a change in fluorescence of the dye coded bead, thereby detecting the target nucleic acid corresponding to each capture oligonucleotide.

20. The method of claim 19, wherein
a. the detectible label is a fluorescent analyte detection dye, the analyte detection dye being excitable by light at a first excitation wavelength and capable of emitting light at a maximum wavelength when excited; and
b. each coded bead has an identification label comprising at least one fluorescent label, the fluorescent identification label being excitable by light of a second excitation wavelength and capable of emitting light at a maximum wavelength, wherein the maximum wavelength of the emitted light of the fluorescent analyte detection dye is different from the maximum wavelength of the emitted light of the fluorescent identification label by at least 80 nm, the first and second excitation wavelengths differ by at least 80 nm, and one of the excitation wavelengths is about 635 nm or greater.

21. The method of claim 20, wherein the identification label comprises at least two fluorescent identification labels in a combination of relative amounts, the fluorescent identification labels being excitable by light of a same second excitation wavelength and capable of emitting lights at maximum wavelengths, distinguishable from each other.

22. The method of claim 19, wherein the capture oligonucleotide comprises a hairpin loop.

23. The method of claim 19, wherein the method is used to detect single nucleotide polymorphisms.

24. The method of claim 19, wherein the nucleic acid cleavage means comprises a structure-specific nuclease.

25. The method of claim 19, wherein the nucleic acid cleavage means comprises a thermostable 5′ nuclease.

26. A method of detecting the presence of a target nucleic acid molecule comprising:
a. providing:
i. a sample comprising at least one target nucleic acid, each target nucleic acid having a first region, a second region and a third region, wherein the first region is contiguous to and downstream from the second region and wherein the second region is contiguous to and downstream from the third region;
ii. a primary cleavage oligonucleotide corresponding to each target nucleic acid, each primary cleavage oligonucleotide having a 3′ portion and a cleavage portion, wherein the 3′ portion of each primary cleavage oligonucleotide comprises a sequence complementary to the third region of each corresponding target nucleic acid and the cleavage portion of each primary cleavage oligonucleotide comprises a sequence complementary to the second region of each corresponding target nucleic acid;
iii. an invading oligonucleotide corresponding to each target nucleic acid, the invading oligonucleotide having a 3′ portion and a 5′ portion, wherein the 3′ portion of each invading oligonucleotide comprises a sequence complementary to the second region of each corresponding target nucleic acid and the 5′ portion of each invading oligonucleotide comprises sequence complementary to the first region of each corresponding target nucleic acid; and
iv. a nucleic acid cleavage means;
b. generating a primary cleavage structure for each target nucleic acid, wherein at least the 3′ portion of the primary cleavage oligonucleotide is annealed to a corresponding target nucleic acid and at least the 5′ portion of the invading oligonucleotide is annealed to a corresponding target nucleic acid, wherein cleavage of the primary cleavage structures are performed by the nucleic acid cleavage means to cleave each primary cleavage oligonucleotide to generate a cleaved assay specific probe corresponding to each target nucleic acid, each cleaved assay specific probe having a 5′ region and a 3′ region;
C. providing a secondary cleavage oligonucleotide for each target nucleic acid, each secondary cleavage oligonucleotide having contiguous first and second regions, wherein the first region comprises the 3′ end of the secondary cleavage oligonucleotide, wherein the second region comprises a region of self-complementarity and a cleavage portion, the cleavage portion being coupled to a detectible label, wherein the first region of each secondary cleavage oligonucleotide is complementary to a cleaved assay specific probe for a corresponding target nucleic acid, and wherein a portion of the second region of each secondary cleavage oligonucleotide is complementary to at least a portion of the 3′ region of the corresponding cleaved assay specific probe;
d. generating a secondary cleavage structure, wherein the cleavage portion of each secondary cleavage oligonucleotide is cleaved from the remainder of the second region of each secondary cleavage oligonucleotide to form a secondary assay specific probe;
e. providing a target-specific reporter oligonucleotide coupled to a coded bead;
f. providing a template oligonucleotide having a first region and a second region, wherein the first region of each template oligonucleotide is complementary to at least a portion of each corresponding secondary assay specific probe, and where the second region of each template oligonucleotide is complementary to the target-specific reporter oligonucleotide;
g. providing a ligator;
h. generating a ligation complex where the first region of each template oligonucleotide is annealed to the corresponding secondary assay specific probe and the second region of each linkage oligonucleotide is annealed to the corresponding target-specific reporter oligonucleotide;
i. ligating the secondary assay specific probe to the corresponding target-specific reporter oligonucleotide to form target-specific detection complexes; and
j. detecting the target-specific detection complexes by detecting a change in the fluorescence of the distinct coded bead, thereby detecting each corresponding target nucleic acid.

27. The method of claim 26, wherein
a. the detectible label is a fluorescent analyte detection dye, the analyte detection dye being excitable by light at a first excitation wavelength and capable of emitting light at a maximum wavelength when excited, and
b. each coded bead has an identification label comprising at least one fluorescent label, the fluorescent identification label being excitable by light of a second excitation wavelength and capable of emitting light at a maximum wavelength, wherein the maximum wavelength of the emitted light of the fluorescent analyte detection dye is different from the maximum wavelength of the emitted light of the fluorescent identification label by at least 80 nm, the first and second excitation wavelengths differ by at least 80 nm, and one of the excitation wavelengths is about 635 nm or greater.

28. The method of claim 27 wherein the identification label comprises at least two fluorescent identification labels in a combination of relative amounts, the fluorescent identification labels being excitable by light of a same second excitation wavelength and capable of emitting lights at maximum wavelengths, distinguishable from each other.

29. The method of claim 26, wherein the method is used to detect single nucleotide polymorphisms.

30. The method of claim 26, wherein the nucleic acid cleavage means comprises a structure-specific nuclease.

31. The method of claim 26, wherein the nucleic acid cleavage means comprises a thermostable 5′ nuclease.

32. A method of detecting the presence of more than one target nucleic acid molecule comprising:
a. providing:
i. a sample comprising at least one target nucleic acid, each target nucleic acid having a first region, a second region and a third region, wherein the first region is contiguous to and downstream from the second region and wherein the second region is contiguous to and downstream from the third region;
ii. a primary cleavage oligonucleotide corresponding to each target nucleic acid, each primary cleavage oligonucleotide having a 3′ portion and a cleavage portion, wherein the 3′ portion of each primary cleavage oligonucleotide is coupled to a solid support, comprises a sequence complementary to the third region of each corresponding target nucleic acid, and is coupled to a detectible label in a first state, and the cleavage portion of each primary cleavage oligonucleotide comprises a sequence complementary to the second region of each corresponding target nucleic acid;
iii. an invading oligonucleotide corresponding to each target nucleic acid, the invading oligonucleotide having a 3′ portion and a 5′ portion, wherein the 3′ portion of each invading oligonucleotide comprises a sequence complementary to the second region of each corresponding target nucleic acid and the 5′ portion of each invading oligonucleotide comprises sequence complementary to the first region of each corresponding target nucleic acid; and
iv. a nucleic acid cleavage means;
b. generating a primary cleavage structure for each target nucleic acid, wherein at least the 3′ portion of the primary cleavage oligonucleotide is annealed to a corresponding target nucleic acid and at least the 5′ portion of the invading oligonucleotide is annealed to a corresponding target nucleic acid, wherein cleavage of the primary cleavage structure is performed by the nucleic acid cleavage means to cleave each primary cleavage oligonucleotide to remove the cleavage portion and change the detectable label to a second state; and
c. detecting cleavage of the primary cleavage oligonucleotides by a change in fluorescence, thereby detecting the target nucleic acid corresponding to the primary cleavage oligonucleotide.

33. The method of claim 32, wherein the solid support attached to the 3′ end of each primary cleavage oligonucleotide comprises a coded bead.

34. The method of claim 33, wherein
a. The detectible label comprises a fluorescent analyte detection dye, the analyte detection dye being excitable by light at a first excitation wavelength and capable of emitting light at a maximum wavelength when excited, and
b. each coded bead has an identification label comprising at least one fluorescent label, the fluorescent identification label being excitable by light of a second excitation wavelength and capable of emitting light at a maximum wavelength, wherein the maximum wavelength of the emitted light of the fluorescent analyte detection dye is different from the maximum wavelength of the emitted light of the fluorescent identification label by at least 80 nm, the first and second excitation wavelengths differ by at least 80 nm, and one of the excitation wavelengths is about 635 nm or greater.

35. The method of claim 34 wherein the identification label comprises at least two fluorescent identification labels in a combination of relative amounts, the fluorescent identification labels being excitable by light of a same second excitation wavelength and capable of emitting lights at maximum wavelengths, distinguishable from each other.

36. The method of claim 34, wherein the fluorescent analyte detection dye comprises a fluorescent reporter and a quencher molecule, the quencher molecule being removed by cleavage of the primary cleavage oligonucleotide.

37. The method of claim 32, wherein the method is used to detect single nucleotide polymorphisms.

38. The method of claim 32, wherein the nucleic acid cleavage means comprises a structure-specific nuclease.

39. The method of claim 32, wherein the nucleic acid cleavage means comprises a thermostable 5′ nuclease.

40. A method of detecting the presence of more than one target nucleic acid molecule comprising:
a. providing:
i. a sample comprising at least one target nucleic acid, each target nucleic acid having a first region, a second region and a third region, wherein the first region is contiguous to and downstream from the second region and wherein the second region is contiguous to and downstream from the third region;
ii. a primary cleavage oligonucleotide corresponding to each target nucleic acid, each primary cleavage oligonucleotide having a 3′ portion and a cleavage portion, wherein the 3′ portion of each primary cleavage oligonucleotide is coupled to a detectible label in a first state and comprises a sequence complementary to the third region of each corresponding target nucleic acid, wherein a 5′ end of the cleavage portion of each primary cleavage oligonucleotide is coupled to a solid support and the cleavage portion comprises one of a fluorescent reporter and a quencher molecule and comprises a sequence complementary to the second region of each corresponding target nucleic acid;
iii. an invading oligonucleotide corresponding to each target nucleic acid, the invading oligonucleotide having a 3′ portion and a 5′ portion, wherein the 3′ portion of each invading oligonucleotide comprises a sequence complementary to the second region of each corresponding target nucleic acid and the 5′ portion of each invading oligonucleotide comprises sequence complementary to the first region of each corresponding target nucleic acid; and
iv. a nucleic acid cleavage means;
b. generating a primary cleavage structure for each target nucleic acid, wherein at least the 3′ portion of the primary cleavage oligonucleotide is annealed to a corresponding target nucleic acid and at least the 5′ portion of the invading oligonucleotide is annealed to a corresponding target nucleic acid, wherein cleavage of the primary cleavage structure is performed by the nucleic acid cleavage means to cleave each primary cleavage oligonucleotide to remove the cleavage portion and change the detectible label to a second state;
c. detecting cleavage of the primary cleavage oligonucleotides by a change in fluorescence, thereby detecting the target nucleic acid corresponding to the primary cleavage oligonucleotide.

41. The method of claim 40, wherein the solid support attached to the 5′ end of each primary cleavage oligonucleotide comprises a coded bead.

42. The method of claim 41, wherein:
a. the detectible label comprises a fluorescent analyte detection dye, the analyte detection dye being excitable by light at a first excitation wavelength and capable of emitting light at a maximum wavelength when excited, and
b. each coded bead has an identification label comprising at least one fluorescent label, the fluorescent identification label being excitable by light of a second excitation wavelength and capable of emitting light at a maximum wavelength, wherein the maximum wavelength of the emitted light of the fluorescent analyte detection dye is different from the maximum wavelength of the emitted light of the fluorescent identification label by at least 80 nm, the first and second excitation wavelengths differ by at least 80 nm, and one of the excitation wavelengths is about 635 nm or greater.

43. The method of claim 42 wherein the identification label comprises at least two fluorescent identification labels in a combination of relative amounts, the fluorescent identification labels being excitable by light of a same second excitation wavelength and capable of emitting lights at maximum wavelengths, distinguishable from each other.

44. The method of claim 42, wherein the fluorescent analyte detection dye comprises a fluorescent reporter and a quencher molecule, the quencher molecule being removed by cleavage of the primary cleavage oligonucleotide.

45. A method of detecting the presence of more than one target nucleic acid molecule comprising:
a. providing:
i. a sample comprising at least one target nucleic acid, each target nucleic acid having a first region, a second region and a third region, wherein the first region is contiguous to and downstream from the second region and wherein the second region is contiguous to and downstream from the third region;
ii. a primary cleavage oligonucleotide corresponding to each target nucleic acid, each primary cleavage oligonucleotide having a 3′ portion and a cleavage portion, wherein the 3′ portion of each primary cleavage oligonucleotide comprises a sequence complementary to the third region of each corresponding target nucleic acid, and the cleavage portion of each primary cleavage oligonucleotide is coupled to a detectible label and a sequence complementary to the second region of each corresponding target nucleic acid;
iii. an invading oligonucleotide corresponding to each target nucleic acid, the invading oligonucleotide having a 3′ portion and a 5′ portion, wherein the 3′ portion of each invading oligonucleotide comprises a sequence complementary to the second region of each corresponding target nucleic acid and the 5′ portion of each invading oligonucleotide comprises sequence complementary to the first region of each corresponding target nucleic acid; and
iv. a nucleic acid cleavage means;
b. generating a primary cleavage structure for each target nucleic acid, wherein at least the 3′ portion of the primary cleavage oligonucleotide is annealed to a corresponding target nucleic acid and at least the 5′ portion of the invading oligonucleotide is annealed to a corresponding target nucleic acid, wherein cleavage of the primary cleavage structure is performed by the nucleic acid cleavage means to cleave each primary cleavage oligonucleotide to remove the cleavage portion;
c. providing a capture oligonucleotide for each primary cleavage oligonucleotide comprising a sequence complementary to the cleavage portion of the primary cleavage oligonucleotide, the capture oligonucleotide being coupled to a dye coded bead; and
d. detecting the cleavage of primary cleavage oligonucleotides by detecting a change in fluorescence of the dye coded bead, thereby detecting the target nucleic acid corresponding to each capture oligonucleotide.

46. The method of claim 45, wherein:
a. the detectible label comprises a fluorescent analyte detection dye, the analyte detection dye being excitable by light at a first excitation wavelength and capable of emitting light at a maximum wavelength when excited; and
b. each coded bead has an identification label comprising at least one fluorescent label, the fluorescent identification label being excitable by light of a second excitation wavelength and capable of emitting light at a maximum wavelength, wherein the maximum wavelength of the emitted light of the fluorescent analyte detection dye is different from the maximum wavelength of the emitted light of the fluorescent identification label by at least 80 nm, the first and second excitation wavelengths differ by at least 80 nm, and one of the excitation wavelengths is about 635 nm or greater; and
c. the fluorescent analyte detection dye comprises a fluorescent reporter and a quencher molecule, the quencher molecule being removed by cleavage of the primary cleavage oligonucleotide.

47. The method of claim 45, wherein the identification label comprises at least two fluorescent identification labels in a combination of relative amounts, the fluorescent identification labels being excitable by light of a same second excitation wavelength and capable of emitting lights at maximum wavelengths, distinguishable from each other.

48. A method of detecting the presence of a target nucleic acid molecule comprising:
a. providing:
i. a sample comprising at least one target nucleic acid, each target nucleic acid having a first region, a second region and a third region, wherein the first region is contiguous to and downstream from the second region and wherein the second region is contiguous to and downstream from the third region;
ii. a primary cleavage oligonucleotide corresponding to each target nucleic acid, each primary cleavage oligonucleotide having a 3′ portion and a cleavage portion, wherein the 3′ portion of each primary cleavage oligonucleotide comprises a sequence complementary to the third region of each corresponding target nucleic acid and the cleavage portion of each primary cleavage oligonucleotide comprises a sequence complementary to the second region of each corresponding target nucleic acid;
iii. an invading oligonucleotide corresponding to each target nucleic acid, the invading oligonucleotide having a 3′ portion and a 5′ portion, wherein the 3′ portion of each invading oligonucleotide comprises a sequence complementary to the second region of each corresponding target nucleic acid and the 5′ portion of each invading oligonucleotide comprises sequence complementary to the first region of each corresponding target nucleic acid; and
iv. a nucleic acid cleavage means;
b. generating a primary cleavage structure for each target nucleic acid, wherein at least the 3′ portion of the primary cleavage oligonucleotide is annealed to a corresponding target nucleic acid and at least the 5′ portion of the invading oligonucleotide is annealed to a corresponding target nucleic acid, wherein cleavage of the primary cleavage structures are performed by the nucleic acid cleavage means to cleave each primary cleavage oligonucleotide to generate a cleaved assay specific probe corresponding to each target nucleic acid, each cleaved assay specific probe having a 5′ region and a 3′ region;
C. providing a secondary cleavage oligonucleotide for each target nucleic acid, each secondary cleavage oligonucleotide having contiguous first and second regions, wherein the first region comprises the 3′ end of the secondary cleavage oligonucleotide, wherein the second region comprises a region of self-complementarity and a cleavage portion, the cleavage portion being coupled to a detectible label, wherein the first region of each secondary cleavage oligonucleotide is complementary to a cleaved assay specific probe for a corresponding target nucleic acid, and wherein a portion of the second region of each secondary cleavage oligonucleotide is complementary to at least a portion of the 3′ region of the corresponding cleaved assay specific probe;
d. generating a secondary cleavage structure, wherein the cleavage portion of each secondary cleavage oligonucleotide is cleaved from the remainder of the second region of each secondary cleavage oligonucleotide to form a secondary assay specific probe;
e. providing a target-specific reporting oligonucleotide coupled to a coded bead;
f. providing a hybridization template oligonucleotide having a first region and a second region, wherein the first region of each hybridization template oligonucleotide is complementary to at least a portion of each corresponding secondary assay specific probe, and where the second region of each template oligonucleotide is complementary to the target-specific reporting oligonucleotide;
g. hybridizing the secondary assay specific probe and the reporter oligonucleotide to the corresponding target-specific hybridization template oligonucleotide to form target-specific detection complexes; and
h. detecting the target-specific detection complexes by detecting a change in the fluorescence of the distinct coded bead, thereby detecting each corresponding target nucleic acid.

49. The method of claim 48, wherein:
a. the detectible label comprises a fluorescent analyte detection dye, the analyte detection dye being excitable by light at a first excitation wavelength and capable of emitting light at a maximum wavelength when excited; and
b. each coded bead has an identification label comprising at least one fluorescent label, the fluorescent identification label being excitable by light of a second excitation wavelength and capable of emitting light at a maximum wavelength, wherein the maximum wavelength of the emitted light of the fluorescent analyte detection dye is different from the maximum wavelength of the emitted light of the fluorescent identification label by at least 80 nm, the first and second excitation wavelengths differ by at least 80 nm, and one of the excitation wavelengths is about 635 nm or greater; and
c. the fluorescent analyte detection dye comprises a fluorescent reporter and a quencher molecule, the quencher molecule being removed by cleavage of the primary cleavage oligonucleotide.

50. The method of claim 48, wherein the identification label comprises at least two fluorescent identification labels in a combination of relative amounts, the fluorescent identification labels being excitable by light of a same second excitation wavelength and capable of emitting lights at maximum wavelengths, distinguishable from each other.

51. A kit for detecting the presence of a target nucleic acid, the kit comprising:
a primary cleavage oligonucleotide corresponding to the target nucleic acid;
a. an invading oligonucleotide corresponding to the target nucleic acid;
b. a nucleic acid cleavage means for cleaving a primary cleavage structure formed by the primary cleavage oligonucleotide, the invading oligonucleotide and the target nucleic acid to form a cleaved assay specific probe;
c. a secondary cleavage oligonucleotide, the secondary cleavage oligonucleotide being coupled to a solid support and a detectible label in a first state and having a portion complementary to at least a portion of the cleaved assay specific probe so that the presence of the cleaved assay specific probe will cause cleavage of a portion of the secondary cleavage oligonucleotide changing the detectible label to a second state.

52. The kit of claim 51 wherein:
a. the solid support is a coded bead; and
b. the detectible label is a fluorescent reporter and quencher molecule, the quencher molecule being removed to change the detectible label to the second state.

53. The kit of claim 52 further comprising a detector for detecting cleavage of the secondary cleavage oligonucleotide by a change in fluorescence, thereby detecting the target nucleic acid corresponding to the secondary cleavage oligonucleotide.
